(19) United States
(12) Patent Application Publication
(10) Pub. No.: US 2008/0014192 A1
(43) Pub. Date: Jan. 17, 2008

(54) Methods For The Diagnosis And Treatment of Fungal Infections Caused By Microorganisms Producing Glucosylceramide

(76) Inventors: Maurizio Del Poeta, Charleston, SC (US); Chiara Luberto, Charleston, SC (US); Talar Kechichian, Glendale, CA (US)

Correspondence Address: FULBRIGHT & JAWORSKI L.L.P., 600 CONGRESS AVE., SUITE 2400, AUSTIN, TX, 78701, US

(21) Appl. No.: 11/752,730

(22) Filed: May 23, 2007

Related U.S. Application Data
(60) Provisional application No. 60/802,678, filed on May 23, 2006.

(51) Int. Cl. A61K 39/395; A61P 43/00; G01N 33/566

(52) U.S. Cl. 424/130.1; 436/501

(57) ABSTRACT

Methods for the diagnosis and treatment of fungal infections caused by microorganisms producing glucosylceramide are provided. In particular, methods for diagnosing or predicting dissemination of a fungal infection from the lungs to the blood stream of a patient are provided. Methods for treating a fungal infection and/or preventing the dissemination of a fungal infection from the lungs to the blood stream or to other organs of a patient, and methods for preventing or treating a fungal infection in a patient by administering a therapeutically effective amount of a fungal glucosylceramide synthase inhibitor to a patient. Such methods are particularly useful for use with patients at risk to develop fungal infections, such as HIV or cancer patients, patients hospitalized in intensive care units, or patients receiving immunosuppressive drugs such as transplant patients.

This application claims benefit of priority to U.S. Provisional Application Ser. No. 60/802,678, filed May 23, 2006, the entire contents of which are hereby incorporated by reference.

This invention was made with government support under grant numbers AI56168 awarded by the National Institute of Allergy and Infectious Diseases (NIAID) of the National Institutes of Health, RR015455 and RR17677 from the National Center for Research Resources of the National Institutes of Health, EPS-0132573 from the National Science Foundation/EPSCoR, and GM069338 from the National Institutes of Health. The United States government has certain rights in the invention.

### BACKGROUND OF THE INVENTION

I. Field of the Invention

The invention relates to the field of molecular biology, particularly to the use of antibodies to glucosylceramide to detect and treat certain fungal infections.

II. Related Art

In most individuals, infections by airborne fungi do not develop into life-threatening diseases. However, in patients hospitalized in intensive care units (ICU's) and in immunocompromised subjects (e.g., HIV-infected, cancer, organ transplant or immunosuppressive drug recipient patients) life-threatening diseases caused by fungal microorganisms are dramatically arising (Calderone and Cihlar (2002) Fungal Pathogenesis, 1-762 (Basel: Marcel Dekker AG, New York)). Current antifungal drugs are often inadequate either because the treatment is initiated too late, recently reviewed in Garber (2001); Stevens (2002); Yeo and Wong (2002); Marr (2003), or because the fungus is resistant due to the limited number of antifungal drugs, recently reviewed in Mukherjee et al. (2005); Rapp (2004).

Thus, new methods for early diagnosis and more effective treatment of fungal infections are needed.

### SUMMARY OF THE INVENTION

Methods for the diagnosis and treatment of fungal infections caused by microorganisms producing glucosylceramide (GlcCer) are provided. Such microorganisms include, for example, Cryptococcus neoformans, Histoplasma capsulatum, Coccidioides immitis, Paracoccidioides brasiliensis, Bastomyces dermatitidis, Sporotrix schenckii, and Aspergillus fumigatus. In particular, methods for diagnosing or detecting fungal infection prior to microbial dissemination from the lungs to the blood stream of a patient are provided. The methods comprise collecting a blood sample from a patient and assaying the sample for the presence of antibodies to fungal GlcCer, where the presence of said antibodies is indicative of an active fungal infection limited to the lung without involvement of other organs of said patient. Methods for treating a fungal infection and/or preventing the dissemination of a fungal infection from the lungs to the blood stream or to other organs of a patient are also provided. These methods comprise administering to a therapeutically effective amount of an antibody to fungal GlcCer to a patient in need thereof. Methods are also provided for preventing or treating a fungal infection in a patient by administering a therapeutically effective amount of a fungal GlcCer synthase inhibitor to a patient. Such methods are particularly useful for use with patients at risk to develop fungal infections, such as HIV or cancer patients, patients hospitalized in intensive care units, or patients receiving immunosuppressive drugs such as transplant patients.

Thus, in accordance with the present invention, there is provided a method for detecting a pulmonary fungal infection prior to dissemination to the brain of an infected patient comprising (a) obtaining a blood sample from said patient; and (b) assaying said sample for the presence of antibodies to fungal glucosylceramide, wherein the presence of said antibodies indicates pulmonary fungal infection. The patient may have been diagnosed with cancer or HIV infection, may be hospitalized in an intensive care unit, or may be receiving immunosuppressive drugs. The assaying may comprise binding of antibodies to glucosylceramide fixed to a support. The bound antibodies may be detected using labeled anti-Ig antibodies, including labels such as an enzyme, a fluorescent label, a chemilluminescent label or a radiolabel. The blood sample may be whole blood, serum or plasma.

The method may further comprise treating said patient for fungal infection; the method may also further comprise culturing a fungal sample from said subject, for example, said fungal sample being typed to be Cryptococcus neoformans, Histoplasma capsulatum, Coccidioides immitis, Paracoccidioides brasiliensis, Bastomyces dermatitidis, Sporotrix schenckii, or Aspergillus fumigatus. The patient may be asymptomatic at the time of assay.

In another embodiment, there is provided a method of assessing the treatment of a patient for fungal infection comprising (a) obtaining a blood sample from said patient; and (b) assaying said sample for the presence and/or level of antibodies to fungal glucosylceramide, wherein the absence or presence of low levels of said antibodies indicates an effective treatment. The patient may have been diagnosed with cancer or HIV infection, is being hospitalized in an intensive care unit, or is receiving immunosuppressive drugs. Assaying may comprise binding of antibodies to glucosylceramide fixed to a support. The antibodies may be detected using labeled anti-Ig antibodies, for example, using anti-Ig labeled with an enzyme, a fluorescent label, a chemilluminescent label or a radiolabel. The blood sample may be whole blood, serum or plasma. The treatment may be an antifungal mono- or combination therapy. The method may be repeated at different time points during said treatment. The treatment may be altered based on the result of step (b), either by changing the dose of a drug or changing from one drug to a different drug.

In yet another embodiment, there is provided a method for treating a fungal infection in a subject comprising administering to said patient a therapeutically effective amount of an IgM or IgG monoclonal antibody to fungal glucosylceramide. The patient may have been diagnosed with cancer or HIV infection, be being hospitalized in an intensive care unit, or be receiving immunosuppressive drugs. The fungal infection may be caused by Cryptococcus neoformans, Histoplasma capsulatum, Coccidioides immitis, Paracoccidioides brasiliensis, Bastomyces dermatitidis, Sporotrix schenckii, or Aspergillus fumigatus.

In still yet another embodiment, there is provided a method for preventing the dissemination of a fungal infection from the lungs to the blood stream or to other organs of a patient comprising administering to said patient in need thereof a therapeutically effective amount of an IgM or IgG monoclonal antibody to fungal glucosylceramide. The patient may have been diagnosed with cancer or HIV infection, be being hospitalized in an intensive care unit, or be receiving immunosuppressive drugs. The fungal infection may be caused by Cryptococcus neoformans, Histoplasma capsulatum, Coccidioides immitis, Paracoccidioides brasiliensis, Bastomyces dermatitidis, Sporotrix schenckii, or Aspergillus fumigatus.

In a further embodiment, there is provided a method for preventing or treating a fungal infection in a patient comprising administering to said patient in need thereof a therapeutically effective amount of a fungal glucosylceramide synthase inhibitor. The patient may have been diagnosed with cancer or HIV infection, be being hospitalized in an intensive care unit, or be receiving immunosuppressive drugs. The fungal infection may be caused by Cryptococcus neoformans, Histoplasma capsulatum, Coccidioides immitis, Paracoccidioides brasiliensis, Bastomyces dermatitidis, Sporotrix schenckii, or Aspergillus fumigatus. The fungal glucosylceramide synthase inhibitor may be a defensin or derivative thereof.

In still a further embodiment, there is provided a method of reducing dissemination of a fungal infection in a patient comprising administering to said patient an agent that depletes alveolar macrophages The fungal infection may be caused by Cryptococcus neoformans, Histoplasma capsulatum, Coccidioides immitis, Paracoccidioides brasiliensis, Bastomyces dermatitidis, Sporotrix schenckii, or Aspergillus fumigatus. The agent may be clodronate, and may be delivered in a lipid vehicle. The method may further comprise administering to said patient a second anti-fungal therapy. The second anti-fungal agent may be an agent that extrudes fungal cells from the intracellular to the extracellular environment, for example, an antimalaric drug.

It is contemplated that any method or composition described herein can be implemented with respect to any other method or composition described herein.

The use of the word “a” or “an” when used in conjunction with the term “comprising” in the claims and/or the specification may mean “one,” but it is also consistent with the meaning of “one or more,” “at least one,” and “one or more than one.” The word “about” means plus or minus 5% of the stated number.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed.

FIGS. 1A-B. Expression of GCS1 genes in S. cerevisiae. (FIG. 1A) In vivo labeling in S. cerevisiae with [³H]-DHS. Expression of cryptococcal and human GCS1 produced GlcCer in galactose (+) but not in glucose (−) (black box). DHS, dihydrosphingosine; S1P, sphingosine-1-phosphate; IPC, MIPC and MIP2C are complex sphingolipids. (FIG. 1B) In vitro assay in S. cerevisiae using NBD-C6-ceramides. Proteins extracted from yeast cells expressing pYES2-HuGCS1 and incubated with NBD-ceramides produced NBD-GlcCer under galactose inducing condition. NBD-GlcCer production was not observed in yeast cells expressing pYES2-CnGCS1 or pYES2 empty vector.

FIGS. 2A-D. (FIG. 2A) In vivo labeling of Cn wild-type (WT), Δgcs1, and Δgcs1+GCS1 using tritiated dihydrosphingosine ([³H]-DHS). The formation of GlcCer (box) was examined by analysis of the extracted lipids onto a TLC. The soy GlcCer standard was visualized by iodine stain. (FIG. 2B) Analysis of purified GlcCer from the cultured strains using HPTLC. The lipids containing the sugar residues were visualized by staining with orcinol in 70% sulfuric acid, and the putative GlcCer is indicated by a black arrow. Plates were also stained with iodine to make sure equal lipid loading among the lanes (black arrowhead). (FIG. 2C) Electrospray tandem mass spectrometric analysis of the glycosphingolipid analyzed on the HPTLC. The regions of the chromatogram indicated in panel B were extracted and analyzed by mass spectrometry as described in the text. The indicated peaks are consistent with the [M+H]⁺ ions for monoexosylceramide with a methyl-sphingadienine backbone and a hydroxy-C18:0 fatty acid (the major species, at m/z 756.7), a non-hydroxy-C18:0 fatty acid (m/z 740.7) and a hydroxy-C16:0 fatty acid (m/z 728.7). (FIG. 2D) ¹H—¹H DQF-COSY (1) and ¹H—¹³C HSQC (2) NMR spectra of the monohexosylceramide WT extracted from the HPTLC. The solid connectivities in (1) between the seven non-exchangeable hexose protons and their ¹H and ¹³C chemical shift characteristics (2) show that glucose is attached to the ceramide backbone, defining this glycosphingolipid as GlcCer. Dashed connectivities in (1) identify the exchangeable 6-OH in 100% dimethyl-sulfoxide-d₆.

FIGS. 3A-B. (FIG. 3A) CBA/J mice infected intranasally with Cn cells. The average survival of mice infected with Cn WT or Δgcs1+GCS1 strain was 24.6±3.9 days or 27.3±4.8 days, respectively. All mice infected with Δgcs1 mutant strain survived after 90 days of infection (P<0.0001). (FIG. 3B) CBA/J mice infected intravenously with Cn cells. The average survival of mice infected with Cn WT or Δgcs1+GCS1 strain was 6.3±0.51 days or 6.2±0.46 days, respectively, whereas the average survival of mice infected with Cn Δgcs1 strain was 15 days (P<0.01).

FIGS. 4A-B. Histopathology of 2 different lungs (FIGS. 4A and 4B) obtained from CBA/J mice infected intranasally with Cn Δgcs1 strain. FIGS. 4A and 4A1, Movat; FIGS. 4B and 4B1, Verhoeff-van Gieson (VVG). FIGS. 4A and 4B, 2.5×; FIGS. 4A1 and 4B1 represents 40× magnification of squared areas in FIGS. 4A and 4B, respectively. In FIG. 4A, white arrowhead indicates Cn cells stained alcian blue; white arrow indicates necrotic tissue; black arrowhead indicates macrophages; black arrow indicates lymphocyte infiltration with fibroblasts and fibrotic tissue. Green arrow indicates normal lung tissue. Black bar=500 μm. In FIG. 4A1, yeast cells (white arrowhead) are found within necrotic tissue. Also, many yeast cells appear as “ghosts” or degenerated cells (yellow arrowhead) within macrophages. Black bar=50 μm. In FIG. 4B, black arrowhead indicates collagen stained red in the peripheral of a nodule containing necrotic tissue, yeast cells, macrophages and lymphocytes. White bar=500 μm. In FIG. 4B1, collagen deposition (red) between lymphocytes and fibrotic tissue. A giant cell in gray (black arrowheads) is loaded with Cn in the internal side of the nodule, surrounded by granulocytes and lymphocytes. White bar=50 μm.

FIGS. 4C-F. Histopathology of 2 different lungs (FIGS. 4C, 9D) obtained from CBA/J mice infected intranasally with Cn Δgcs1 strain, and 2 different lungs (FIGS. 4EL and 4FL) and 2 different brains (FIGS. 4EB and 4FB) obtained from CBA/J mice infected intravenously. FIGS. 4C, 4C1, 4EL and 4FL, hematoxylin and eosin; FIGS. 4D, 4D1, 4EB and 4FB, mucicarmine. FIGS. 4C, 4D, 4EL, 4FL, 4EB and 4FB, 10×; FIGS. 4C1 and 4D1 represents 40× magnification of squared areas in FIGS. 4C and 4D, respectively. In FIG. 4C, white arrow indicates lymphocyte infiltration in proximity of a macrophage aggregation (square). Green arrow indicates normal lung. White bar=200 μm. In FIG. 4C1 (hematoxylin and eosin), Cn cells (white arrowhead) are readily recognized within a large macrophage. Other macrophages contain “ghosts” or degenerated yeast cells (yellow arrowhead). White bar=50 μm. In FIG. 4D, lymphocyte and macrophage (white arrows) infiltration in proximity of Cn engulfed within a giant macrophage, readily appreciated in FIG. 4D1. Green arrow indicates normal lung tissue. In FIG. 4D, black bar=200 μm. In FIG. 4D1, white bar=50 μm. In FIGS. 4EL and 4FL, Cn cells are localized in small nodules with a host cellular infiltration comprised by lymphocytes and neutrophils (black arrows). Green arrows indicate normal lung. In FIGS. 4EB and 4FB, Cn Δgsc1 cells are almost exclusively contained within brain abscess (black arrowheads) with the absence of a granulomatous response. In FIGS. 4EL, 4FL, 4EB and 4FB, black bar=200 μm.

FIGS. 5A-D. GlcCer is required for growth of Cn cells in 5% CO₂ at pH 7.4. Cn WT, Δgcs1, and Δgcs1+GCS1 strains were grown in DMEM medium at 37° C. in 5% CO₂ at pH 7.4 (FIG. 5A) or pH 4.0 (FIG. 5B);* P<0.001, Δgcs1 versus WT. In FIG. 5C, cell cycle analysis of Cn strains after 24 h of incubation in DMEM medium at 37° C. in 5% CO₂ at pH 7.4.* P<0.01, Δgcs1 versus WT. In FIG. 5D, illustration of the succession of biological events characterizing a yeast cell cycle.

FIGS. 6A-D. (FIG. 6A) A diagram illustrating the deletion of Cn GCS1 gene using pΔgcs1 plasmid cassette. (FIG. 6B) Southern analysis of genomic DNA digested with HindIII (H3) of wild-type (WT) and 5 transformants, using the indicated probes. Transformant #42 showed homologous recombination with double cross over event without any ectopic or loop integration. This strain was designated Cn Δgcs1. (FIG. 6C) Reintroduction of GCS1 gene into the Cn Δgcs1 strain using pCR-GCS1-HYG plasmid cassette. (FIG. 6D) Southern analysis of genomic DNA digested with EcoRI (RI) extracted from wild-type (WT) Δgcs1 and transformant #14, which shows a single crossover event at the 5′-UTR plus insert of plasmid loop with consequent insertion of a second GCS1 copy. This strain was designated Δgcs1+GCS1.

FIG. 7. NMR analysis. The ¹H and ¹³C chemical shifts (ppm) from ¹H, ¹³C-heteronuclear single quantum correlation (HSQC) experiment and measured vicinal ³J_{HH} coupling constants from Double Quantum Filtered-COrrelation SpectroscopY (DQF-COSY) experiment of hexose attached to ceramide correspond to glucose (Koerner et al. (1987); Dabrowski (1994)).

FIG. 8. Lung tissue burden culture of CBA/J mice infected intranasally with Cn wild-type (WT) or Δgcs1 strain during the course of infection. Lungs infected with Cn wild-type show a progressive increase number of yeast cells and, eventually, mice will succumb by day 20-28 of infection (please see FIG. 3B). Lungs infected with Δgcs1 strain show a ˜500-fold decrease in the number of yeast cells by day 7 and ˜100-fold decrease during the remaining time of infection compared to the initial inoculum.

FIG. 9. Intracellular growth. Five hours of post-incubation of J774.16 macrophage-like cells with Cn WT, Δgcs1 or Δgcs1+GCS1, the number of macrophages containing yeast cells was determined (phagocytic index) and buds were counted only in yeast cells inside macrophages (% budding/phagocytic index). Counts are geometric means ±standard deviation of at least 8 different fields.

FIGS. 10A-D. Virulence factors. The ability to grow at 37° C. and ambient CO₂ (FIG. 11A), production of polysaccharide capsule (FIG. 11B) and melanin formation (FIG. 11C) are not affected by lack of Gcs1/GlcCer. (FIG. 11D) Cn Δgcs1 mutant exerts a growth defect on 0.05% SDS agar plate compared to WT or reconstituted strains but not in 1M NaCl, or in presence of nitric oxide or hydrogen peroxide.

FIG. 11. Level of anti-glucosylceramide (anti-GlcCer) antibody in sera (used at 1:24 dilution) obtained from HIV patients with cryptococcosis (red) and normal subjects (blue) using an ELISA assay.

FIGS. 12A-C. Survival studies on murine models infected with Cn strains. (FIG>12A) All CBA/J mice infected with Cn Δgcs1 survived significantly longer (90 days) than either WT or Δgcs1+GCS1 (* P<0.0001). (FIG. 12B) Tgε26 mice infected with WT or Δgcs1+GCS1 survived an average of 24.6±3.9 days and 27.3±4.8 days, respectively. Conversely, the Δgcs1 infected mice survived 58.5±6.2 days (†, P<0.001). (FIG. 12) Summary of the average survival of each group ±SD.

FIGS. 13A-B. Effect of clodronate treatment on lung AMs. (FIG. 13A) AM count in the mouse broncho alveolar lavage 48 hours after treatment with PBS, empty liposomes, or clodronate showed a significant AM decrease in clodronate-treated mice (* P<0.001). (FIG. 13B) AM count in mouse broncho alveolar lavage 7 days after treatment with PBS, empty liposomes or clodronate. Clodronate caused a significant decrease in AMs (†, P<0.05). Results are expressed as number of AM's X10⁴ per mouse.

FIGS. 14A-B. (FIG. 14A) Survival studies done on Tgε26 mice receiving weekly doses of anesthesia alone, PBS, empty liposomes or clodronate containing liposomes. The mice were infected with Cn WT or Δgcs1. As summarized in (FIG. 14B) while no significant change is seen among the different groups infected with Cn WT, clodronate-treated mice survived an average of 76.6±5.3 days (†, P<0.05) significantly longer than controls.

FIGS. 15A-D. Viable Cn in internal organs of Tgε26 mice that were treated with PBS, empty liposomes or clodronate and infected intranasally with Cn WT. No significant differences in the number of colony forming units was observed in lung (FIG. 15A), spleen (FIG. 15C), or kidney (FIG. 15D) homogenates at different times points after intranasal infection with Cn WT. Clodronate treated mice showed a reduction of tissue burden culture in the brain (FIG. 15B) but this difference was not statistically significant. Results are expressed as colony forming units per organ on a logarithmic scale.

FIGS. 16A-D. Viable Cn in internal organs of Tgε26 mice that were treated with PBS, empty liposomes or clodronate and infected intranasally with Cn Δgcs1. Growth and dissemination of the Δgcs1 strain in the lungs (FIG. 16A), brain (FIG. 16B), kidney (FIG. 16C) and spleen (FIG. 16D) is retarded in the clodronate-treated group. * P<0.05.

FIGS. 17A-F. Histopathology of lungs infected with Δgcs1 strain. (FIG. 17A) Empty liposome-treated lung at day 16 of infection; black arrows indicate intracellular Δgcs1 cells. Black bar=50 μm. (FIG. 17B) Clodronate-treated lung at 16 days of infection; black arrows indicates an intracellular Cn whereas green arrows indicate extracellular Δgcs1 cells. Black bar=50 μm. (FIG. 17C) Empty liposome treated lung at 36 days of infection; black and green arrows indicate Δgcs1 cells intra and extracellularly. Black bar=400 μm. (FIG. 17D) Clodronate-treated lung at 36 days of infection; green arrows indicate few Δgcs1 cells, yellow arrows indicate the limited infiltration of granulocytes in the lung parenchyma and in the bronchioli. Black bar=400 μm. (FIG. 17E) Empty liposome treated lung at 50 days of infection; Δgcs1 cells infiltrate the lung parenchyma (green arrows) and lymph nodes (red arrows) with destruction of the tissue. Black bar=400 μm. (FIG. 17F) Clodronate-treated lung at 70 days of infection. Δgcs1 cells are almost exclusively localized in the lung parenchyma. The lack of Cn cells in the alveolar spaces (black arrows) is remarkable. Granulocyte infiltrations are indicated by yellow arrows. Black bar=400 μm.

FIGS. 18A-B. Histopathology of 2 different lungs (FIGS. 18A and 18B) obtained from CBA/J mice infected intranasally with Cn Δgsc1 strain. (FIG. 18A) Movat stain; (FIG. 18B) Verhoeff-van Gieson (VVG) stain. In FIG. 18A, white arrowhead indicates Cn cells stained alcian blue; white arrow indicates necrotic tissue; black arrowhead indicates macrophages; black arrow indicates lymphocyte infiltration with fibroblasts and fibrotic tissue. Green arrow indicates normal lung tissue. Black bar=500 μm. In FIG. 18B, black arrowhead indicates collagen stained red in the periphery of a nodule containing necrotic tissue, yeast cells, macrophages and lymphocytes. White bar=500 μm.

FIGS. 19A-H. Histopathology of 3 mucicarmine-stained lungs obtained from Tgε26 mice infected intranasally with Cn Δgsc1. (FIGS. 19A and B) Lungs treated with empty liposome at 16 days after infection. Black arrows indicate Cn cells within macrophages. Black bar=50 μm. (FIG. 19C) Infiltration of lymph node by Cn gsc1 cells in mice treated with empty liposomes 16 days after infection. Black bar 200 μm. (FIG. 19D) Magnification of FIG. 19C showing few Cn cells within giant macrophages. Black bar=50 μm. (FIGS. 19E and 19F) Clodronate-treated lungs at 16 days after infection. Green arrows indicate Cn cells localized extracellularly, whereas black arrows indicate Cn cells localized intracellularly. Black bar=200 μm. (FIGS. 19G and 19H) Clodronate treated lungs at 36 days after infection. Very few Cn cells are observed and they are mostly extracellular. Black bar=50 μm.

FIG. 20. Production of monoclonal antibody IgM against GlcCer. 7 mice were infected with 10³ cells of Cn wild-type H99 and 2 mice with 10³ cells of Δgcs1. At the illustrated time points blood from the safenous vein was withdrawn and examined for the presence of IgM anti-GlcCer antibody. At day 29 two mice and at day 57 one mouse infected with Cn wild-type were sacrificed. Lung, brain, liver, spleen and kidney were removed. Spleen was fused to a myeloma cell line (to make monoclonal antibody) whereas other organs were tested for the presence of fungal cells by colony forming units (CFU). Cn cells were found in the lung but not in the kidney, liver or brain. At day 70, 80 and 90, the remaining mice were sacrificed and organs tested for CFU. Mice infected with Cn wild-type showed CFU in brains whereas mice infected with Δgcs1 did not show any CFU in brain or other organs. Spleen from mice infected with Δgcs1 was fused to a myeloma cell line as a negative control for antibody production.

FIG. 21. IgM anti-GlcCer in sera from patients in which the Cn antigen resulted positive or negative

### DETAILED DESCRIPTION OF THE INVENTION

The present invention provides methods for the diagnosis and treatment of fungal infections caused by microorganisms producing glucosylceramide (GlcCer), including, for example, Cryptococcus neoformans, Histoplasma capsulatum, Coccidioides immitis, Paracoccidioides brasiliensis, Bastomyces dermatitidis, Sporotrix schenckii, and Aspergillus fumigatus. In particular, methods are provided for diagnosing or detecting a fungal infection prior to dissemination from the lungs to the brain of a patient. Methods are also provided for treating a fungal infection and/or preventing the dissemination of a fungal infection from the lungs to the blood stream, brain or to other organs of a patient.

The studies discussed below show that antibodies against GlcCer are detected using an ELISA assay in mouse sera when Cn is still in the lung. The inventors reasoned that since GlcCer is a fungal molecule required for Cn survival in the lung environment, its production would be up-regulated. This up-regulation may trigger the host to stimulate an immune response through the production of antibodies anti-GlcCer. Eventually, in a mouse animal model, Cn will then disseminate from the lung to the brain, suggesting that naturally induced anti-GlcCer antibodies are not fully capable of stopping the progression of cryptococcosis. Regardless of the potential efficacy of anti-GlcCer as a therapeutic mean, preliminary studies clearly suggest that antibody anti-GlcCer can be used as an early diagnostic method for cryptococcal infection and as a predictor of Cn dissemination in the bloodstream and in the brain.

### I. GLUCOSYLCERAMIDE

One of the cell surface molecules of Cryptococcus and other fungi is glucosylceramide (GlcCer), an antigenic glycosphingolipid that elicits an antibody response in patients affected with cryptococcosis (Rodrigues et al., 2000) and in mice (Toledo et al., (2001). GlcCer is synthesized by UDP-glucose:ceramide glucosyltransferases encoded by GlcCer synthase (GCS) genes, which catalyze the first step in glycosphingolipid biosynthesis leading to the formation of the membrane lipid GlcCer. Very little is known about the role and function of GlcCer synthase in fungal cells. The synthesis of GlcCer is absent in the yeast Saccharomyces cerevisiae, but it has been demonstrated in other fungi such as Pichia pastoris, in fungi pathogenic to plants, such as Magnaporthe grisea (Leipelt et al., 2001, and to humans, such as Cn (Rodrigues et al., 2000, Candida albicans (Leipelt et al., 2001; Matsubara et al., 1987; and Leipelt et al., 2000, Aspergillus fumigatus (Boas et al., 1994; Levery et al., 2002, Histoplasma capsulatum (Toledo et al., 2001, Paracoccidioides brasiliensis (Takahashi et al., 1996, Sporotrix schenckii (Toledo et al., 2001, and other fungi (reviewed in (Warnecke et al., 2003; Hanada, 2005; and Heung et al., 2006). The gene encoding for GlcCer synthase (GCS1) has been isolated from P. pastoris, M. grisea, and C. albicans (Leipelt et al., 2001). The GCS1 gene has been deleted in the pathogenic fungus C. albicans, but its role in the pathogenicity of this fungus has yet to be elucidated.

Other studies suggested a role for GlcCer in the regulation of fungal growth and pathogenesis. For instance, in Cn GlcCer is mainly localized in the cell wall and mostly accumulates at the budding site of dividing cells. Interestingly, antibodies against Cn GlcCer produced by patients affected with cryptococcosis inhibit budding and division of Cn cells grown in vitro (Rodrigues et al., 2000), and differentiation and germ-tube formation of Pseudallescheria boydii and C. albicans (Pinto et al., 2002). Additionally, production of antibody against fungal glycolipids has been also demonstrated in patients with paracoccidioidomycosis (Toledo et al., 1995), although in this disease the antibodies are mostly interacting with galactosylceramide instead of GlcCer. In other fungi, disruption of the GlcCer biosynthetic pathway altered spore germination, hyphal development and fungal growth (Levery et al., 2002; Thevissen et al., 2004). Monoclonal antibodies against fungal GlcCer have been produced (Toledo et al., 2001) and, interestingly, treatment with anti-GlcCer antibody enhanced macrophage function against the fungus Fonsecaea pedrosoi (Nimrichter et al., 2004).

Altogether, these studies may suggest an important role for GlcCer in fungal cell growth and differentiation and, because germ-tube formation and hyphal growth are pro-virulent factors, it also suggests that GlcCer might be implicated in the regulation of fungal virulence. Interestingly, Gcs1 and GlcCer are found in a variety of pathogenic fungi and because a significant biochemical difference between human and Cn Gcs1 exists, compounds that would specifically inhibit the fungal enzyme represent a novel class of antifungal agents against fungal infections (Levery et al., 2002; Thevissen et al., 2004). Furthermore, since antibodies against the fungal GlcCer inhibit Cn growth in vitro and they do not interact with the human GlcCer (Toledo et al., 2001), they represent a possible therapeutic approach to control infections due by fungal microorganisms producing GlcCer.

As described in greater detail below, GlcCer is required for the development of the fungal associated-meningo-encephalitis. A mutant strain lacking the enzyme responsible for production of GlcCer (GlcCer synthase) cannot grow and leave the lung of the host (disseminate) and reach the brain when it enters the body through inhalation (see Example 1 below). These studies show that the loss of pathogenicity is due to the fact that, once in the alveoli, the fungal GlcCer is required for the microorganism to replicate in the host lung environment, which is characterized by alkaline pH and high concentrations of carbonic dioxide (CO₂). These biological studies reveal that, under these environmental conditions, GlcCer promotes the transition of the fungal cell cycle, which is an essential process for cell replication.

### II. FUNGI AND PATIENTS AT RISK

Two medical developments in the mid-1900's resulted in a major increase in the number of fungal diseases: the clinical use of corticosteroids and the discovery of antibacterial drugs. However, only in the last 20 years have fungal diseases have become a significant threat to human health. As potent antibacterial drugs were widely administered in empirical fashion, and a variety of foreign body devices were inserted into critically ill patients, these patients became susceptible to fungal diseases. Medical advances in management of cancers with chemotherapy and the management of organ transplantations with potent immunosuppressive agents have unintended increase the population at risk of infection due to yeast- and mold-related diseases. The advent of the human immunodeficiency virus (HIV) pandemic has resulted in an even larger number of patients at risk for fungal diseases. There is simply no location in the world in which a dramatic rise in invasive mycoses has not occurred. This in turn has resulted in a terrible toll of morbidity, mortality and costs taken on the human population. Three major invasive fungal infections afflict immunocompromised subjects—cryptococcosis, aspergillosis and candidiasis—each of which are discussed below.

In addition, there are many other fungal microorganisms producing GlcCer that, in addition to infect immunocompromised patients, they also infect immunocompetent subjects. Among others, these microorganisms include Blastomyces dermatitidis, Histoplasma capsulatum, Coccidioides immitis, Paracoccidioides brasiliensis, Fonsecaea pedrosoi, and Sporotrix schenckii. Nothing is known on the role of GlcCer in the pathogenesis of these microbes. However, it is quite interesting that IgG antibodies against cryptococcal GlcCer cross-react with the yeast but not with the mold form of H. capsulatum, P. brasiliensis and S. schenckii (Toldeo et al., 2001). Since it is the yeast form of the latter fungi that is invasive and not the mold form, the inventors hypothesize that the production of anti-GlcCer during the course of these infections may predict their dissemination.

A. Cryptococcosis

Cryptococcosis is a chronic human disease caused by the environmental fungus Cryptococcus neoformans (Cn), which enters the body through the respiratory tract. Even though normal subjects can be infected by Cn, the majority of the cases of cryptococcosis occurs in immunocompromised patients where the fungus leaves the lung and disseminates to the brain causing the most common meningo-encephalitis worldwide. Prior to the mid-1950's, fewer than 300 cases of cryptococcosis had been reported in the medical literature, but today it ranks as one of the most common infection disease agents to cause human meningoencephalitis. A study in 1992, during the peak of the AIDS epidemic, showed that several urban areas had cryptococcosis rates that soared to over 5 cases per 100,000 persons (Hajjeh et al., 1999). Then, during the wide spread of the highly active antiretroviral therapy (HAART) in the mid- to late 1990s, rates of systematic infections were substantially reduced in the United States to approximately 1 case per 100,000 person per year, which is comparable to the incidence rate of another meningeal pathogen, Neisseria meningitides (van Elden et al., 2000). However, in less-developed countries in sub-Saharan Africa and Asia in which HAART is not readily available, 15-45% of HIV patients are affected by cryptococcosis with a mortality rate of 100% within two weeks if untreated and still has 25% mortality even with antifungal treatment (Gumbo et al., 2002; Hakim et al., 2000; Heyderman et al., 1998).

In addition, to AIDS, numerous other immunodeficient conditions predispose to disseminated cryptococcosis. Cn infection has been associated with lymphoproliferative malignancies. For instance, the incidence of Cn infection among patients suffering from chronic lymphatic leukemia, Hodgkin's disease, chronic myelogenous leukemia and multiple myeloma was 24.3, 13.3, 10.9, and 6.9, respectively, per 1,000 patients (Kaplan et al., 1977). The median overall survival of patients with lymphoproliferative disorders affected by cryptococcosis is 2 months, which is significantly shorter than the 9 months median survival of an AIDS patient with cryptococcosis (White et al., 1992). Cryptococcosis is also associated with organ transplantation. Renal transplant recipients appear to be at particular risk and several studies have reported a prevalence of cryptococcosis in renal transplant patients of 3-4% (Kohno et al., 1994; Shaariah et al., 1992). In more recent studies, Cn infection was documented in 2.8% or organ transplant recipients with an overall death of 42% (Husain et al., 2001).

Infection initiates upon inhalation of spores or desiccated yeast. In the lung, the fungus proliferates in the alveolar space and in immunocompetent subjects the infection is normally contained in this organ. However, in immunocompromised subjects, dissemination of the yeast cells from the lung to the brain can occur intra- and extra-cellularly, leading to the development of a life-threatening disease (Feldmesser et al., 2001; Levitz et al., 1999; and Chretien et al., 2002). Since Cn is a facultative intracellular pathogen, many studies have addressed how the fungus survives and grows within host cells (Feldmesser et al. (2001) Trends Microbiol., 9:273-278; Feldmesser et al. (2000) Infect. Immun., 68:4225-4237; Lee et al. (1995) Lab. Invest., 73:871-879; Tucker and Casadevall, 2002; Steenbergen et al., 2001; and Luberto et al., 2001) but how fungal cells survive and proliferate extracellularly is not well understood.

Although there are many methods and assays available for the diagnosis of cryptococcosis, such as the India ink and other staining methods of body fluids for the direct observation of Cn cells, detection of Cn capsular antigen in body fluids by a latex agglutination test, and culture assays, these methods can only detect a disseminated cryptococcal disease only when the fungus already spread out from the lung. The latex agglutination test for serum cryptococcal antigen has been proposed as a screening test in endemic area to identify patients at risk of developing cryptococcosis. However, studies have shown that in asymptomatic patients with HIV infection, a periodic antigen test did not identify or predict infection that occurred only 4 to 8 months after a negative antigen screening test was obtained (Negroni et al., 1995). This is actually not surprising because this test detects a component of the Cn cell capsule. Thus, in order for this Cn antigen to be detected in the serum, a Cn cell(s) must be present in the bloodstream and, as such Cn dissemination already occurred. Interestingly, serological antibodies (IgG and IgA) against Cn antigen have been observed in sera of infected patients but they have poor value as a diagnostic method because these antibodies are often absent in asymptomatic patients (Speed et al., 1996).

B. Aspergillosis

Invasive aspergillosis is an increasingly common infection caused by an environmental fungus Aspergillus spp. An unselected autopsy series documented a 132% increase in invasive aspergillosis between 1978-92 (Groll et al., 1996) and another center reported a 3-fold increase in cumulative annual incidence between 1990-98 (Marr et al., 2002). The overall incidence of invasive aspergillosis is estimated at 1 to 2:100,000 population per year in the U.S. (Rees et al., 1998), greater than the annual US incidence of meningococcal infection or of listeriosis (Center for Diseases, 2002). The annual death rate from invasive aspergillosis has increased by >350% between 1980-97 in the U.S. to ˜1:100,000 population (McNeil et al., 2001), a figure comparable to the annual U.S. deaths from asthma (National Institute of Health, 1995). Although there have been notable recent advances in diagnosis and therapy, the case-fatality rate of invasive aspergillosis was 58% in the latest systematic review of the literature (Lin et al., 2001), and mortality rates ranged from 30-69% in recent large studies (Bowden et al., 2002; Herbrecht et al., 2002; Schwartz et al., 2005) with all patients dying of the infection while receiving appropriate treatment.

The isolation of the fungus in the lung by bronco-alveolar lavage or biopsy is often required to make a diagnosis of aspergillosis. During invasive aspergillosis in which the fungus disseminates through the bloodstream to other organs and tissues, the presence in the serum of Aspergillus antigen(s), such as galactomannan, suggest the diagnosis but the sensitivity of this test is low in the early disease, and, as for the Cn antigen, this test detects a fungal particle and, as a consequence, it is presumable that Aspergillus cells should be present in the serum. Antibodies against Aspergillus antigens are produced during invasive aspergillosis but no standardized tests exist for these antibodies (Bennett et al., 2003). In the inventors' studies, they found that their IgM monoclonal antibodies made against Cn GlcCer cross-react with GlcCer extracted from Aspergillus fumigatus (Af). Thus, the GlcCer produced by Af is antigenically similar to that one produced by Cn. As such, the hypothesize that upon Af infection of the lung, the host will produce IgM against Af GlcCer that can be detected by the same ELISA assay as for Cn.

C. Candidiasis

Candida is the most common cause of nosocomial fungal infections, accounting for 8% of hospital-acquired septicemias. More than 12% of bone marrow transplant patients develop candidiasis with a mortality rate of 50-80%, even after appropriate treatment. In patients with prolonged granulocytopenia, endogenous Candida spp. found in the GI tract will pass through the GI mucosa as a prelude to vascular invasion and hematologic dissemination (Hopfer and Amjadi, 2002). The traditional methods of detection of candidiasis have been shown be to insensitive. The diagnosis of invasive candidiasis rely almost exclusively on the detection of fungal cells in the bloodstream by yeast culture, a method that takes 2-5 days to become positive. This contributes to the grim nature of invasive candidiasis since almost 50% of candidial fatalities occur within the first week of fungal dissemination (Hopfer and Amjadi, 2002). Other diagnostic tests include antibody (IgG or IgM) and antigen detection. However, most of the antigen assays have sensitivity <75% and serum IgG or IgM against Candida antigens is often not used because the patient population in which invasive candidiasis occurs is highly immunosuppressed. Whether high-level of fecal IgA could be used to predict the dissemination of fungal cells from the GI into the bloodstream is largely unknown. Interestingly, fecal IgA can be detected in immunocompromised patients, probably because their production and secretion is regulated locally at the GI mucosa (Wamey et al., 1994).

Candida spp. produce GlcCer that causes an extremely serious fungal invasive disease that normally do not enter the body through inhalation but through the skin, and the gastro-intestinal (GI) and the urinary tracts. Candida spp. produce GlcCer and, thus, antibody against GlcCer should also be found in sera of patients that eventually develop invasive candidiasis. If GlcCer is required for Candida fungal cells to be infectious in body sites other than the lung, such as the skin, the gastro-intestinal and the urinary tracts, then it is expected that a patient with anti-GlcCer may also have the potential to develop invasive candidiasis. In this case, one will expect that these patients would develop IgA anti-GlcCer and not IgM, particularly if GlcCer would be required for the dissemination of Candida cells from the GI tract into the bloodstream. Although GlcCer has been found in Candida spp. (Warnecke and Heinz, 2003), nothing is known about its pathobiological role in the development Candida infections.

### III. DIAGNOSTICS

According to the inventors' findings, discussed below, production of GlcCer is increased during the lung infection process and, thus, the host would respond with the production of antibody, as this lipid is immunogenic. Thus, the presence of antibody anti-GlcCer, previously shown to be produced (Rodrigues et al., 2000), would be detectable at high-level particularly during the initial phase of the fungal infection process before the microorganisms would reach the blood stream and other organs and before the manifestation of clinical signs and symptoms of disseminated disease. As a consequence, testing for the presence of these antibodies in blood could be an early diagnostic method for infections that have yet to begin the dissemination process, or that have just disseminated to the blood stream. The identification of this initial asymptomatic phase is important because it could prompt the administration of an antifungal therapy that would prevent the development not only of neurological disease caused by Cn infection, but also life-threatening disease caused by other fungi such as Histoplasma capsulatum, Coccidioides immitis, Paracoccidioides brasiliensis, Bastomyces dermatitidis, Sporotrix schenckii, and Aspergillus fumigatus. Although all these fungi produce GlcCer, the presence of antibody in sera during the infection against glycolipids has been investigated only in patients affected with cryptococcosis and paracoccidioidomycosis.

Accordingly, in one embodiment of the present invention, a method for diagnosing or detecting dissemination of a fungal infection from the lungs to the blood stream of a patient is provided, comprising collecting a blood sample from a patient and assaying the sample for the presence of antibodies to fungal GlcCer, where the presence of such antibodies is indicative of lung fungal infection prior to the microbial dissemination from the lungs to the blood stream of the patient. In particular embodiments, the fungal infection is caused by Cryptococcus neoformans, Histoplasma capsulatum, Coccidioides immitis, Paracoccidioides brasiliensis, Bastomyces dermatitidis, Sporotrix schenckii, or Aspergillus fumigatus.

B. Preparing Antibodies

Methods for the production of antibodies against fungal GlcCer are well known in the art, as described in Toledo et al. (2001), and Nimrichter et al. (2004), as well as references cited throughout the present specification and incorporated herein by reference.

C. Immunologic Assays

Antibodies of the present invention can be used in characterizing the GlcCer antibody content of blood/serum/plasma in subjects through techniques such as RIAs, ELISAs and Western blotting. This provides early detection of fungal infections, possibly prior to onset of dissemination.

In the present invention, an ELISA assay is particularly contemplated. For example, GlcCer may be immobilized onto a selected surface, preferably a surface exhibiting a affinity for this molecule. After washing to remove incompletely adsorbed material, it is desirable to bind or coat the assay plate wells with a non-specific agent that is known to be antigenically neutral with regard to the test sera, e.g., bovine serum albumin (BSA), casein or solutions of powdered milk. This allows for blocking of non-specific adsorption sites on the immobilizing surface and thus reduces the background caused by non-specific binding of antibody to antigen on the surface.

After binding of antibody to the well, coating with a non-reactive material to reduce background, and washing to remove unbound material, the immobilizing surface is contacted with the sample to be tested in a manner conducive to immune complex (antigen/antibody) formation.

Following formation of specific immunocomplexes between the test sera and the GlcCer, and subsequent washing, the occurrence and even amount of immunocomplex formation may be determined by subjecting the same to a second antibody having specificity for the test antibody. Appropriate conditions preferably include diluting the sample with diluents such as BSA, bovine gamma globulin (BGG) and phosphate buffered saline (PBS)/Tween®. These added agents also tend to assist in the reduction of non-specific background. The detecting antibody is then allowed to incubate for from about 2 to about 4 hr, at temperatures preferably on the order of about 25° to about 27° C. Following incubation, the surface is washed so as to remove non-immunocomplexed material. A preferred washing procedure includes washing with a solution such as PBS/Tween®, or borate buffer.

To provide a detecting means, the second antibody will preferably have an associated enzyme that will generate a color development upon incubating with an appropriate chromogenic substrate. Thus, for example, one will desire to contact and incubate the second antibody-bound surface with a urease or peroxidase-conjugated anti-human IgG for a period of time and under conditions which favor the development of immunocomplex formation (e.g., incubation for 2 hr at room temperature in a PBS-containing solution such as PBS/Tween®).

After incubation with the second enzyme-tagged antibody, and subsequent to washing to remove unbound material, the amount of label is quantified by incubation with a chromogenic substrate such as urea and bromocresol purple or 2,2′-azino-di-(3-ethyl-benzthiazoline)-6-sulfonic acid (ABTS) and H₂O₂, in the case of peroxidase as the enzyme label. Quantitation is then achieved by measuring the degree of color generation, e.g., using a visible spectrum spectrophotometer.

Other potential labels include radiolabels, fluorescent labels, dyes and chemilluminescent molecules (e.g., luciferase).

D. Titers and Significance

As can be seen in Table 1, below, the minimum threshold titer that the present assay can detect is quite low (>1:20,000 dilution). Any negative results (OD=O) of a serum diluted at 1:192 would thus be considered a “true negative” result. All values above this number are positive, meaning that an OD at 5 or 10 with a dilution at 1:192 will be considered positive for antibody against GlcCer, and thus a clinician would deem such subject as in need of an anti-fungal therapy.

Generally, a low, medium and high level classification can be used, with values (at 1:192 dilution) of low (OD=0-33), medium (OD=33-66) and high (OD=66-100). Of course, these numbers will vary with further serum dilutions, for example, an OD of 33 is a much higher value if the serum has been diluted 1:20,000 compared to a OD value of 33 obtained with a serum diluted only 1:192.

The higher the titer, the more aggression is the infection and the more potent the patient response. Lower antibody levels, but nonetheless positive, are indicative of infection prior to dissemination.

### IV. TREATMENTS

In other embodiments of the present invention, methods for treating a fungal infection and/or preventing the dissemination of a fungal infection from the lungs to the blood stream, brain or other organs of a patient are also provided. These methods comprise administering a therapeutically effective amount of an antibody to fungal GlcCer to a patient in need thereof. Treatment with antibody against GlcCer has been shown to inhibit growth of fungal cells in vitro (Rodrigues et al., 2000; Nimrichter et al., 2004) and because GlcCer is required for fungal growth in the lung as described above and in Example 1 below. In particular embodiments, the fungal infection is caused by Cryptococcus neoformans, Histoplasma capsulatum, Coccidioides immitis, Paracoccidioides brasiliensis, Bastomyces dermatitidis, Sporotrix schenckii, or Aspergillus fumigatus.

A. Pharmaceutical Formulations

As used herein, by an “effective” amount or a “therapeutically effective amount” of a drug or active agent (e.g., an antibody or antifungal drug) is meant a non-toxic but sufficient amount of the drug or agent to provide the desired effect, i.e., reducing or eliminating fungal cell growth, differentiation, or dissemination. It is recognized that the effective amount of a drug or active agent will vary depending on the route of administration, the selected drug or active agent, and the species to which the drug or pharmacologically active agent is administered. It is also recognized that one of skill in the art will determine appropriate effective amounts by taking into account such factors as metabolism, bioavailability, and other factors that affect plasma levels of the drug or active agent. A drug or active agent may be administered by any route of administration sufficient to achieve the desired effect, including by aerosol or intravenous administration.

B. Antifungal Agents

A variety of antifungal agents have been approved or are in development. These include the allylamines (amorolfine, butenafine, naftifine, terbinafine), anti-metabolites (flucytosine, fluconazole, intraconazole, ketoconazole, posaconazole, ravuconazole), azoles (voriconazole, clotrimazole, econazole, miconazole, oxiconazole, sulconazole, terconazole, tioconazole), chitin synthase inhibitors (nikkomycin Z, caspofungin), glucan synthase inhibitors (micafungin, anidulafungin, amphotericin B, AmB Lipid Complex, AmB Colloidal Dispersion), polyenes (Liposomal AmB, AmB oral suspension, liposomal nystatin, topical nystatin, pimaricin), other systemics (griseofulvin, ciclopirox olamine) and other topicals (haloprogrin, tolnaftate, undecylenate).

C. Glucosylceramide Synthase Inhibitors

In other embodiments of the present invention, methods are provided for preventing or treating a fungal infection in a patient by administering a therapeutically effective amount of a fungal GlcCer synthase inhibitor to a patient. There are peptides produced by plants and insects called defensins that interact with fungal lipid GlcCers and lead to the killing of fungi in vitro (Thevissen et al., 2004). Accordingly, in one embodiment of the invention, fungal GlcCer synthase inhibitors for use in the methods of the present invention for preventing or treating a fungal infection in a patient include defensins.

Although there are molecules and compounds that inhibit the mammalian glucosylceramide synthase enzyme (reviewed in Shayman and Abe, 2000), these compounds do not inhibit the fungal enzyme, which has a different substrate specificity compared to the mammalian enzyme (see Example 1 below). These results suggest that their modes of action are different and, thus, a selective compound targeting one (fungal) but not the other (mammalian) has a highly likelihood to kill the fungus without having toxic effects on mammalian cells. Accordingly, in one embodiment of the invention, fungal GlcCer synthase inhibitors for use in the methods of the present invention for preventing or treating a fungal infection in a patient selectively inhibit fungal GlcCer synthase but not mammalian GlcCer synthase.

The methods of the present invention for preventing or treating a fungal infection are particularly useful for patients at risk to develop fungal infections, such as HIV or cancer patients, patients hospitalized in intensive care units, or patients receiving immunosuppressive drugs such as transplant patients.

In further embodiments of the present invention, methods are provided for the high throughput screening of compounds or molecules that specifically target the fungal GlcCer synthase enzyme and not the mammalian counterpart enzyme. Such methods are well known in the art, reviewed in Clearly et al. (2005), and Raventos et al. (2005).

D. Alveolar Macrophage Depleting Agents

In order to prevent or limit fungal disease, it may be desirable to reduce alveolar macrophage counts in a subject. One alveolar macrophage depleting agent is clodronate (dichloromethylenediphosphonic disodium salt), typically used to treat a high level of calcium in the blood caused by changes in the body that occur with cancer. Clodronate also treats the weakening in the bones when cancer has spread to the bones from another part of the body. It is available in oral and parental forms. The oral form is 1600 mg to 2400 mg given in one or two divided amounts per day, no more than 3200 mg in a day. It should be taken at least two hours before or after food. For parental administration, 300 mg in a solution is injected over at least two hours into a vein once a day for two to five days. The treatment should not be longer than seven days. The amount of medicine may be less if kidney problems are present.

E. Combination Therapies

Pathogen resistance to drugs represents a major problem in modern medicine. One solution to this problem is to combine various therapies to achieve added and sometimes synergistic effects. In the context of the present invention, it is contemplated that treatment with the disclosed antibody preparations, glucosylceramide synthase inhibitors and alveolar macrophage depleting agents could be used in conjunction with traditional antifungal interventions.

To kill, inhibit growth, inhibit spread, or otherwise reverse or reduce the pathogenic phenotype of fungi, using the methods and compositions of the present invention, one would generally administer an GlcCer antibody, glucosylceramide synthase inhibitors and/or alveolar macrophage depleting agents and at least one other agent. These compositions would be provided in a combined amount effective to achieve the beneficial results set forth above. This process may involve contacting the cells with both agents at the same time. This may be achieved by contacting the cell with a single composition or pharmacological formulation that includes both agents, or by contacting the cell with two distinct compositions or formulations, at the same time.

Alternatively, the antibody, glucosylceramide synthase inhibitor and/or alveolar macrophage depleting agent treatment may precede or follow the other agent treatment by intervals ranging from minutes to weeks. In embodiments where the other agent and the antibody are applied separately to the cell, one would generally ensure that a significant period of time did not expire between the time of each delivery, such that the agent and the antibody, glucosylceramide synthase inhibitor and/or alveolar macrophage depleting agent would still be able to exert an advantageously combined effect on the cell. In such instances, it is contemplated that one would administer both modalities within about 12-24 hours of each other and, more preferably, within about 6-12 hours of each other, with a delay time of only about 12 hours being most preferred. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations.

It also is conceivable that more than one administration of either the antibody or the other agent will be desired. Various combinations may be employed, where the antibody therapy, glucosylceramide synthase inhibitor and/or alveolar macrophage depleting agent is “A” and the other agent is “B”, as exemplified below:

| | | | | | | | |
|---|---|---|---|---|---|---|---|
| A/B/A | B/A/B | B/B/A | A/A/B | B/A/A | A/B/B | B/B/B/A | B/B/A/B |

| | | | | | | |
|---|---|---|---|---|---|---|
| A/A/B/B | A/B/A/B | A/B/B/A | B/B/A/A | B/A/B/A | B/A/A/B | B/B/B/A |
| A/A/A/B | B/A/A/A | A/B/A/A | A/A/B/A | A/B/B/B | B/A/B/B | B/B/A/B |

Other combinations are contemplated. For example, in the inventors' studies (see Examples 1 and 2), they found that GlcCer is required for extracellular replication of fungal cells. But in addition to growing extracellularly (e.g., alveolar spaces, bloodstream), many fungi including Cn do survive and grow intracellularly within the phagolysosome of macrophages. Thus, although an anti-GlcCer treatment will be very effective in reducing fungal replication in the extracellular environment, it would not be able to remove the intracellular fungal component. Interestingly, antimalaric drugs, such as chloroquine, are found to promote extrusion of fungal cells from the intracellular to the extracellular compartment (Ma et al., 2006 and Alvarez et al., 2006). Thus, treatment with an antimalaric drug will force Cn cells to be extracellularly and, thus, they will potentiate the effect of an anti-GlcCer inhibitor. In general, this is a crucial aspect in immunocompromised patients in which fungal microorganisms reside and replicate also within macrophages (see Example 3). Importantly, the implementation of such combination therapy (antimalaric plus and anti-GlcCer antibody or compound) will be extremely useful in certain regions, such as Africa, in which cryptococcosis is highly associated with HIV infection in the same areas where malaria is endemic. Thus, by taking antimalaric drugs and an anti-GlcCer inhibitor, these HIV patients would be highly protected from cryptococcosis.

Agents or factors suitable for use in a combined therapy are those set forth above. It also should be pointed out that any of the foregoing therapies may prove useful by themselves in treating a fungal infection.

### V. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

The pathogenic fungus Cryptococcus neoformans (Cn) infects humans upon inhalation and causes the most common fungal meningo-encephalitis in immunocompromised subjects worldwide. In the host, Cn is found both intracellularly and extracellularly, but how these two components contribute to the development of the disease is largely unknown. Here we show that a glycosphingolipid, glucosylceramide (GlcCer), present in Cn is essential for fungal growth in host extracellular environments, such as in alveolar spaces and in the bloodstream, which are characterized by a neutral/alkaline pH, but not in the host intracellular environment, such as in the phagolysosome of macrophages, which is characteristically acidic. Indeed, a Cn mutant strain lacking GlcCer cannot grow in vitro at neutral/alkaline pH whereas has no growth defect at acidic pH. The mechanism by which GlcCer regulates alkali tolerance is by allowing the transition of Cn through the cell cycle. This study establishes Cn GlcCer as a key virulence factor of cryptococcal pathogenicity, with important implications for future development of new antifungal strategies.

A. Methods

Strains, plasmids, and growing conditions. The strains used in this study were Cryptococcus neoformans var. grubii serotype A strain H99 (WT), Δgcs1 mutant derived from H99, and Δgcs1+GCS1 reconstituted strain derived from Δgcs1, the Saccharomyces cerevisiae strain JK9-3dα (MAT-α, trp1, leu2-3, his4, ura3, ade2, rme1) and JK9-3dα containing the pYES2, or pYES2-HuGCS1 or pYES2-CnGCS1. Escherichia coli strains DH5-α (Invitrogen) and TOP10 (Invitrogen) were used in this study. Plasmids pBluescript SK (Stratagene), pCR2.1-TOPO (Invitrogen) were used for cloning, and pYES2 (Invitrogen) was used for expression of GlcCer synthase genes in S. cerevisiae. Cn strains were routinely grown in yeast extract/peptone/2% dextrose (YPD) rich medium. S. cerevisiae strain JK9-3dα transformed with the pYES2 or pYES2HuGCS1 or pYES2CnGCS1 vector was routinely grown in synthetic medium containing 6.7 g/liter Yeast Nitrogen Base (YNB) without amino acids, 1.2 g/liter of amino acid mixture lacking uracil, and 2% glucose or 1% glucose and 1% galactose for 16 h at 30° C. to induce either human or cryptococcal GCS1 gene expression. A full length cDNA clone of the human GCS1 gene was cloned into pYES2 (Invitrogen) vector as described (Ichikawa et al., 1996), generating the pYES2-HuGCS1 plasmid. Bacterial strains for plasmid amplification were routinely grown at 37° C. in Luria-Bertani (LB) medium containing 75 mg/liter of ampicillin (Sigma). DMEM (Difco #11995) medium buffered with Hepes at pH 7.4 or pH 4.0 was used for growing Cn strain at 37° C. in presence of 5% CO₂, as described.

Isolation and cloning of the Cn GCS1 gene. The GCS1 gene from Cn was identified by a BLAST search of the human GCS sequence (GenBank D50840) in the genome database of Cn var. grubii serotype A strain H99 from Duke Center for Genome Technology (cneo.genetics.duke.edu/blast.html). One putative sequence with E value of 9e-51 corresponding to chr4Bpiece18 was found. The GCS1 cDNA was then isolated by RT-PCR using the following primers: PRGCS-5′Fw (5′-ATG AGC GAT TCC GGC ACA TTG TCC-3′) and PRGCS3′Rv (5′-TCA ATT TCT ATC ACC CAA ACG AATTG-3′) and total RNA extracted from Cn strain H99. A 1353 bp fragment was obtained, cloned into the pCR2.1-TOPO vector generating plasmid pCRCnGCS1, and sequenced. Amino acid alignment of Cn Gcs1 with other Gcs1 proteins from different organisms revealed a homology (identity+similarity) of 47%, 16%, 43%, 46%, 43%, 35%, to human, Arabidopsis thaliana, Candida albicans, Pichia pastoris, Magnaporthe grisea, and Aspergillus nidulans, respectively. The Cn Gcs1 contains the motifs (D1, D2, D3, (R/QXXRW)) common to other GCSs required for GCS activity (Marks et al., 2001), and the characteristic N-terminus hydrophobic transmembrane domain, which contains a signal-anchor sequence to the Golgi outer membrane (Ichikawa et al., 1996). The Cn GCS1 cDNA sequence was deposited in the GenBank database (AY956317). The GCS1 gene was deleted and reconstituted in Cn var. grubii (WT) generating Cn Δgcs1 and Δgcs1+GCS1 strains.

In vitro growth studies. From overnight YPD broth cultures of Cn WT, Δgcs1 and Δgcs1+GCS1 strains, cells were washed twice in SDW, resuspended, and diluted into 40 ml fresh YPD broth to a final density of 10⁴ cells/ml and incubated in a shaker incubator at 30° C. or 37° C. Aliquots were taken at indicated time points and serial dilutions were plated onto YPD plates for assessment of colony forming units (CFU). For in vitro growth studies on DMEM at pH 7.4, 7.0 or 4.0, 10⁴ cells/ml were incubated at 37° C. in presence of 5% CO₂ and aliquots taken at indicated time points and serial dilutions were plated onto YPD plates for assessment of CFU. Capsule formation and melanin production were examined using media as previously described (Casadevall and Perfect (1998). For cell wall stability, cells were spotted in serial dilutions on YPD plates containing 0.05% SDS. For osmotic stress, cells were spotted in serial dilutions on YPD plates 1M of NaCl. For oxidative or nitrosative stresses, cells were spotted in serial dilutions on YNB plates containing amino acids adjusted to pH 4.0 and supplemented with H₂O₂ to a final concentration of 5 mM or nitrite to a final concentration of 1 mM. After incubation at 30° C. or 37° C. for 72 h, cell growth was examined and recorded by photography. Results show plates incubated at 37° C.

For intracellular growth studies, J774.16 macrophage-like cells were used and fungal growth of Cn WT, Δgcs1 and Δgcs1+GCS1 strains was determined as previously described (Feldmesser et al. (2001) Trends Microbiol., 9:273-278; Feldmesser et al., 2000; Lee et al., 1995; Tucker and Casadevall, 2002; Steenbergen et al., 2001; and Luberto et al., 2001).

For cell cycle studies, yeast cells were incubated in DMEM pH 7.4 at 37° C. in presence of 5% CO₂ for 24 h and 36 h. Then, cells were washed twice with ice-cold water, sonicated, and fixed in 7 ml of 95% ethanol for 16 h. After fixing, the cells were then pelleted, washed with 5 ml of 50 mM sodium citrate, resuspended in 1 ml of 50 mM sodium citrate containing 0.25 mg/ml of RNase A and incubated at 50° C. for 1 h. Next, proteinase K was added at a final concentration of 20 mg/ml and cells were kept at 50° C. for an additional 1 h. Finally, 1 ml of 50 mM sodium citrate containing 50 μg/ml of propidium iodine was added. Cells were kept 24 h in the dark at 4° C. Cells were analyzed using Becton Dickinson fluorescence-activated cell analyzer. Data were modeled using the program ModFit LT V3.1.

In vitro activity assay of glucosylceramide synthase. To express Cn GCS1 gene in S. cerevisiae, the cDNA fragment was digested with EcoRI from pCR-CnGCS1 and subcloned into EcoRI-restricted pYES2 plasmid, generating pYES2-CnGCS1. The S. cerevisiae JK9-3dα strain was chemically transformed with pYES2-CnGCS1 plasmid. Transformants were selected on YNB ura⁻ agar plates containing 2% glucose, randomly chosen, purified, and stored. For gene induction, cell cultures were pelleted and washed twice with SDW. Cells were resuspended and inoculated into YNB ura⁻ broth containing 1% galactose+1% glucose or 2% glucose and incubated in a 30° C. shaker for 16 h. For in vitro GCS activity in Cn strains, yeast cells were grown on YPD media in a shaker incubator for 24 h at 30° C., and yeast cells were harvested by centrifugation, washed with SDW, and pellets stored at −80° C., if not used immediately. Once cells were collected, proteins were extracted as previously described (Luberto et al., 2001) and quantitated by the method of Bradford (1976). The in vitro GCS activity assay was performed by using the fluorescent ceramide analog 7-nitro-2-1,3-benzoxadiazol-4-yl (NBD)-C6-ceramide (Avanti Polar Lipids) as a substrate and monitoring the formation of NBD-C6-glucosylceramide as described previously (Ichikawa et al., 1996) with some modifications. Briefly, 200 μg of yeast proteins were incubated for 30 min at 30° C. in 50 mM bis-Tris-HCl buffer (pH 6.5) containing 12.5 mM KCl, 250 μM EDTA, 50 μM phosphatidylcholine, 500 μM UDP-Glc, 5 mM Triton X-100, 20 μM NBD-C6-ceramide in a final reaction volume of 100 μL. The reaction was terminated by the addition of 0.3 ml of CHCl3:MeOH (1:1). The samples were mixed and the phases separated by centrifugation at 2000×g for 5 min. The lower phase was dried down using the SPD 2010 Speedvac® (Thermosavant). The chloroform-soluble product was analyzed by analytical thin layer chromatography (TLC) on silica gel 60 plates (EM Science) using the solvent system chloroform/methanol/water (65:25:4). Purified GlcCer from soy (Avanti Polar Lipids) was also loaded onto the TLC plate and used as a standard (Sullards et al., 2000). NBD-GlcCer was identified as a fluorescent band migrating in the correspondence of the soy GlcCer and quantitated by direct fluorescence using a phosphoimager Molecular Dynamics 840 STORM unit and by ImageQuant analysis.

In vivo activity assay of glucosylceramide synthase. Approximately 5 h before the cell collection, yeast cells were counted and 5×10⁷ cells were resuspended in the same growing media and incubated with 2 μM/ml of NBD-C6-ceramide. Similar experiments were performed using NBD-C6 phytoceramide, NBD-C12-ceramide and NBD-C12-phytoceramide. Yeast cells were incubated for 5 h shaking at 30° C. Cells were harvested by centrifugation for 10 min at 2000×g, washed with SDW and the total lipid was extracted using the method of Mandala (Mandala et al., 1995) followed by base hydrolysis. The extracted lipids were loaded onto a TLC using the solvent system chloroform:methanol:water (65:25:4). Formation of NBD-GlcCer was analyzed as described above.

For in vivo labeling using tritiated dihydrosphingosine ([³H]-DHS), ˜2 h prior cell collection, 5×10⁷ yeast cells/ml were incubated with 2 μCi/ml of [³H]-DHS for 2 h at 30° C. in the appropriate growing media in shaker incubator. The cells were harvested by centrifugation (10 min at 2500×g), washed with SDW and stored at −80° C. if not used immediately. Lipids were extracted according to Mandala (1995) and, after base hydrolysis, loaded onto a TLC plate, which were analyzed using a phosphoimager and quantitation of radioactive GlcCer performed by ImageQuant analysis. As a control, 10 μl of 20 mM of soy glucosylceramide obtained from Avanti Polar Lipids was loaded onto a TLC plate and visualized using iodine stain.

Mass spectrometry analysis. Purified GlcCer was obtained as described in supplementary materials and used for mass spectrometry analysis. The MS and MS/MS scans of GlcCer were conducted using an API 3000 triple quadrupole mass spectrometer with electrospray ionization as described in Merrill et al. (Merrill et al., 2005). The analyses used syringe infusion and included neutral loss scans (with m/z 180 for loss of the hexose headgroup) and precursor ion scans for the characteristic product ion m/z 276.4.

Nuclear Magnetic Resonance (NMR) analysis. NMR experiments were recorded in 500 ml dimethyl-sulfoxide-d₆ (CIL, Andover, Mass.) using a 160 mg of putative monohexosylceramide obtained from HPTLC purification; subsequently, the sample was dissolved in dimethyl-sulfoxide-d₆ and deuterium oxide (98:2, v/v). NMR spectra were recorded on a Varian Inova 600 MHz spectrometer equipped with an actively shielded z-gradient triple-resonance (¹H, ¹³C, ³¹P) probe at 25° C. All spectra were processed and analyzed using FELIX 2000 (MSI, San Diego, Calif.) and NMRPipe (Delaglio et al., 1995) program packages. ¹H chemical shifts were internally referenced to TSP. Carbon-13 chemical shifts were referenced against the center line of the dimethyl-sulfoxide septet (d=39.52 ppm). The DISPI-2 mixing time was 62.6 ms (Shaka et al., 1988). Difference NOE experiments were measured with saturation delays of 6 sec (45).

Virulence studies and histology analysis in a murine mouse model of cryptococcosis. Four- to six-week-old CBA/J (NCI/Frederick Laboratories) mice were used. Mice were anesthetized with an intraperitoneal injection of 60 μl of xylazine/ketamine mixture, containing 95 mg of ketamine per kilogram of body weight and 5 mg of xylazine per kilogram of body weight. Cn wild-type, Δgcs1 and Δgcs1+GCS1 strains were prepared by growing cells for 24 h at 30° C. in YPD medium. The cells were pelleted, washed twice, and resuspended in phosphate buffered saline (PBS) pH 7.4 at a concentration of 2.5×10⁷ cells/ml. Ten CBA/J mice were infected with 5×10⁵ cells of wild-type, Δgcs1 or Δgcs1+GCS1 strains in a volume of 20 μl through nasal inhalation. Eight CBA/J mice were infected with 10⁶ cells of wild-type, Δgcs1 or Δgcs1+GCS1 strains in a volume of 100 μl through the tail vein injection. The mice were fed ad libitum and followed with twice-daily inspections. Mice that appeared moribund or in pain or with clinical sign of meningo-encephalitis were sacrificed using CO₂ inhalation followed by cervical dislocation. All animal procedures are approved by the Institutional Animal Care and Use Committee (IACUC) and follow the guidelines of the American Veterinary Medical Association. Histology analysis was performed as described in supplementary materials.

Statistical Analysis. Survival data from the mice experiment were analyzed by Kluskal-Wallis test. For other statistical analysis, a Student's t test was used. A P value of less than 0.05 was considered to be significant.

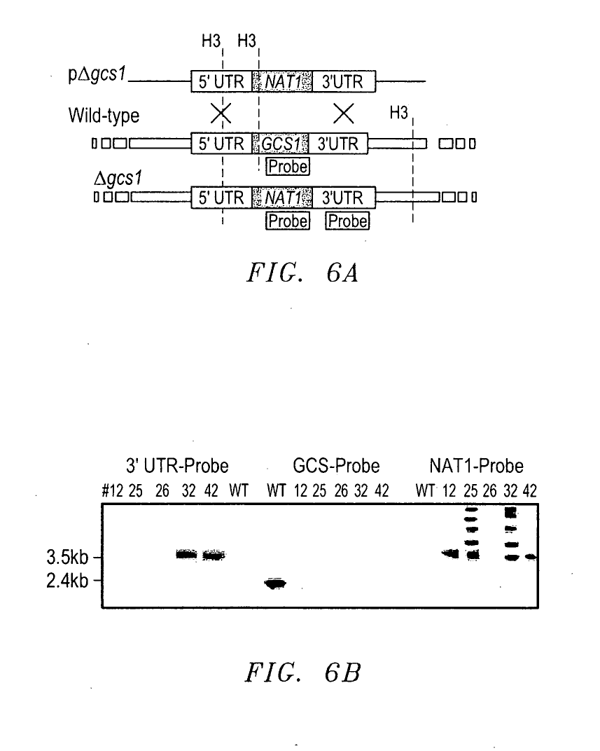

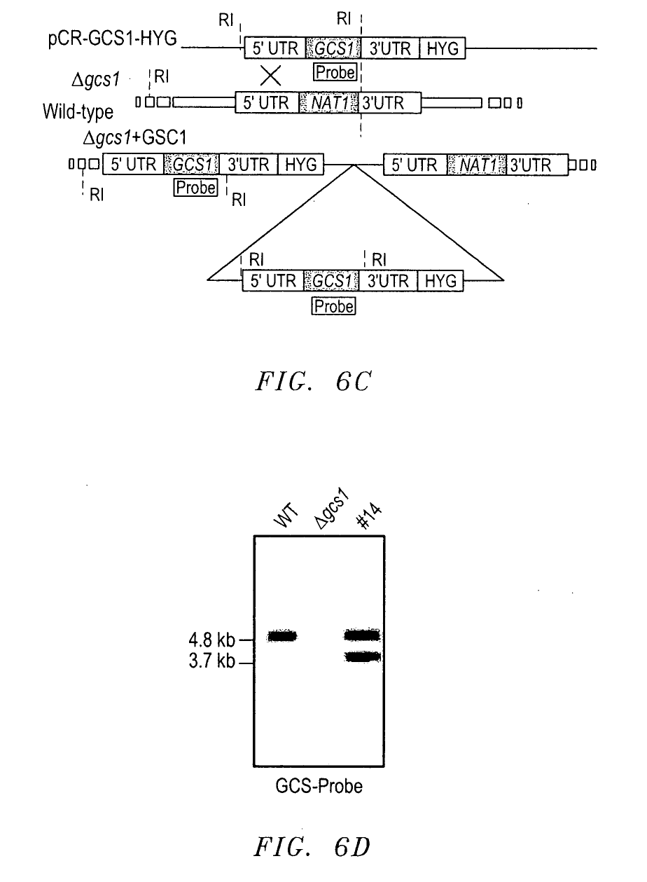

Deletion of Cn GCS1 gene and reconstitution. To delete the GCS1 gene from the genome of Cn a plasmid construct was created that contained 1.5 kb of the 5′ untranslated region (5′-UTR) upstream the open reading frame (ORF) as well as 1.5 kb of 3′-UTR flanking the nourseothricin acetyltransferase gene (NAT1) gene, whose expression is under the control of actin promoter producing yeast cells resistant to the antibiotic nourseothricin (Werner BioAgents). The 5′-UTR was generated by PCR using genomic DNA as a template and primers: PRUTR1Sac 5′-CTG GAGCTC CGA AGT AAA GGC TGG CTT AGC TGA-3′ and PRUTR1Spe 5′-GAG ACTAGT ACC TAT GAA GGG AAT GAA TAT TGC-3′, which contain SacI and SpeI sites (bold and underlined), respectively. The 3′-UTR region was generated by PCR using H99 genomic DNA as a template and primers PRUTR2Fw 5′-GAG AGATCT TTT GGT TTT CAA AGG CTC TGC ATG-3′ and PRUTR2Rev 5′-GAG GGTACC TAT ATC ACC GCT CAA TAA TAG CTT-3′, which contain BglII and KpnI sites (bold and underlined), respectively. The resulting fragments were cloned into the pCR2.1-TOPO vector generating plasmids pCR-5UTR and pCR-3UTR and sequenced. The plasmid pCR-5UTR was digested with SacI and SpeI and the resulting 1.5 kb fragment was subcloned into the SacI/SpeI-restricted pCR-NAT1 vector, generating plasmid pCR-5UTR:NAT1. The pCR-NAT1 was created as previously described (Heung et al., 2004; Heung et al., 2005). Plasmid pCR-3UTR was digested with KpnI and XhoI and the 1.5 kb 3′-UTR fragment was subcloned into a KpnI-XhoI restricted pBluescript SK vector generating plasmid pSK-3UTR. Finally, plasmid pCR-5UTR:NAT1 was digested and the 3.2 kb fragment corresponding to the 5UTR-NAT1 was subcloned into pSK-3UTR vector, generating plasmid pSK-5UTR-NAT1-3UTR which was re-named pΔgcs1 (FIG. 6A).

The Cn wild-type strain H99 was transformed with plasmid pΔgcs1 using biolistic delivery of DNA, according to (Toffaletti et al., 1993). Transformants were grown on YPD plates containing 100 μg/ml of nourseothricin. Colonies were chosen randomly and purified. Genomic DNA preparation for Southern blot analysis was performed according to (Sambrook et al., 1989). Five transformants showing deletion of the GCS1 gene and insertion of the plasmid cassette were obtained and transformant #42 was chosen and designated Cn Δgcs1 strain (FIG. 6B).

To reintroduce the GCS1 gene back in the Δgcs1 mutant we generated the pCR-GCS1-HYG plasmid construct as follow (FIG. 6C): 1) Fragment A (4.7 kb) containing the entire GCS1 ORF and 1.5 kb of the upstream (5′UTR) and downstream (3′UTR) regions were generated by PCR using H99 genomic DNA as a template and primers PRUTR1Sac 5′-CTG GAGCTC CGA AGT AAA GGC TGG CTT AGC TGA-3′ and PRUTR2Rev 5′-GAG GGTACC TAT ATC ACC GCT CAA TAA TAG CTT-3′, containing a SacI and KpnI site, respectively (bold and underlined). This 4.7 kb fragment was cloned into the pCR2.1-TOPO vector generating plasmid pCR-GCS1. 2) Fragment B (2.5 kb) containing the hygromycin B gene (HYGB) conferring resistance to hygromycin B (Calbiochem #400051) was obtained by digesting the pCnTel vector (Cox et al., 1996) using XbaI and HindIII. This fragment was blunted and cloned into SpeI restricted-blunted pCR-GCS1 vector, generating pCR-GCS1-HYG construct. The Cn Δgcs1 mutant was transformed with pCR-GCS1-HYG plasmid using biolistic delivery of DNA. Transformants were grown on YPD plates containing 100 μg/ml of hygromycin B. Stable transformants were selected, grown on YPD, DNA extracted and three transformants showed re-introduction of wild-type GCS1 gene and the introduction of a second GCS1 copy by the insertion of the plasmid loop. Transformant #14 was chosen and designated Cn Δgcs1+GCS1 (FIG. 6D).

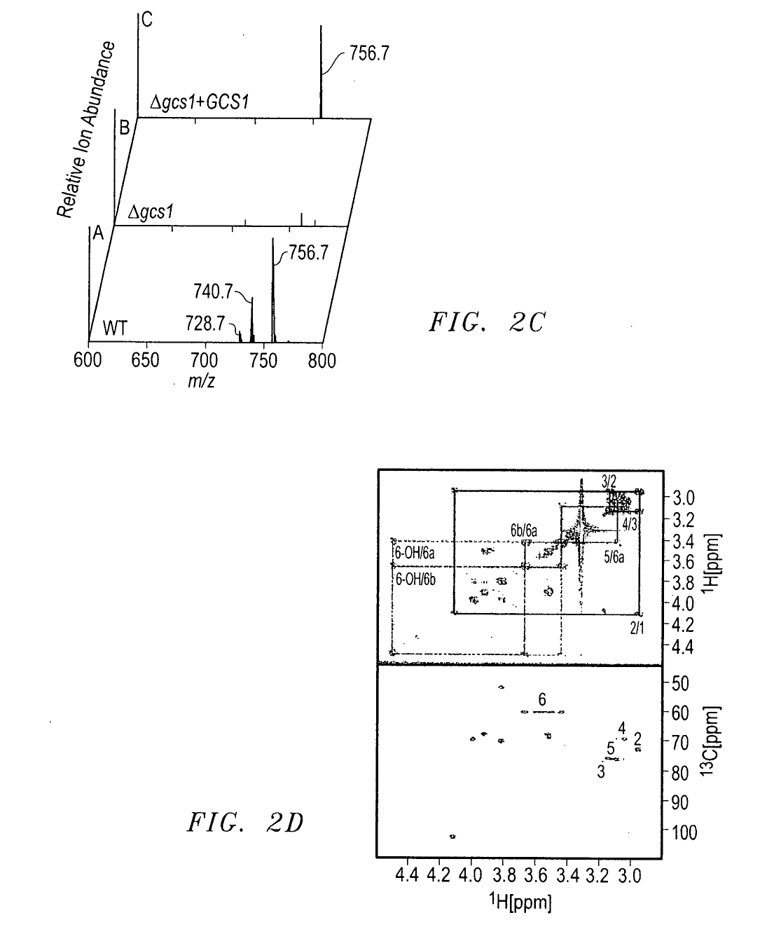

Purification of GlcCer. ClcCer was purified from Cn cells following the protocol described in (Rodrigues et al., 2000). Briefly, Cn wild-type, Δgcs1 and Δgcs1+GCS1 strains were grown on YPD media in a shaker incubator for 48 h at 30° C. The cells were washed twice with SDW and 6 pellets of 5×10⁸ cells per strain were resuspended in 1 ml total lipid extraction (TLE) buffer (95% ethanol:SDW:diethylether:pyridine:14.8N NH₄OH-15:15:5:1:0.018) and incubated for 30 min at 60° C. with brief vortexing. After centrifugation for 10 min at 2000×g the supernatants of two samples were combined and dried down. Dried pellets were suspended in 2 ml methanol, 1 ml chloroform was added and samples were incubated for 30 min at 37° C. with brief vortexing. After centrifugation for 10 min at 2000×g, the supernatant was transferred to another tube, 1 ml chloroform and 1 ml H₂O were added, and the phases were homogenized by vortexing twice for 30 sec. Then, phases were separated by centrifugation for 5 min at 2500×g and the lower phase was dried down. The pellet was suspended in 1 ml chloroform:acetic acid (99:1) and loaded into a Sep-Pak® Cartridge (Waters) previously equilibrated with 15 ml chloroform. Neutral lipids fraction were eluted with 15 ml chloroform:acetic acid (99:1) and discarded. Glycolipid-fraction was eluted with 10 ml acetone and dried down. Pellets were suspended in 0.5 ml chloroform and 0.5 ml of 0.6 M KOH in methanol, incubated for 1 h at room temperature, neutralized with 0.325 ml of 1 M HCl, and phases were separated by adding 0.125 ml of H₂O. The organic phase was transferred into a new tube, dried down and resuspended in 1 ml chloroform:acetic acid (99:1). The mixture was then re-loaded into a Sep-Pak® Cartridge (Waters) previously equilibrated with 15 ml chloroform. The column was sequentially eluted with 15 ml chloroform:acetic acid (99:1), 10 ml chloroform:methanol (95:5), 15 ml chloroform:methanol (9:1), and 10 ml chloroform:methanol (8:2), 5 ml chloroform:methanol (1:1) and 5 ml methanol. The fractions eluted with the chloroform:methanol ratio of 9:1 would contain GlcCer and were dried down for high performance thin layer chromatography (HPTLC) analysis. These lipid fractions were suspended in chloroform:methanol (2:1) and spotted on a Kieselgel 60 (HPTLC-Merck) and developed in the solvent chloroform:methanol/H₂O (65:25:4). Plates were dried at room temperature and the sugar residues were stained by repeated spraying of the plate with 0.2 mg/l orcinol/70% H₂SO₄ and heating for 15 min in an oven at 100° C. To make sure that equal amount of lipid extracts were loaded in each lane, plates were also exposed to iodine, which revealed that approximately an equal amount of lipids were loaded (arrowhead in FIG. 2B).

Histology analysis. Lung, brain, liver, kidney and spleen of CBA/J mice infected with the above strains were collected. Organs were fixed in 37% formaldehyde (Sigma), embedded in paraffin, and stained with hematoxylin and eosin to visualize the host inflammatory response, mucicarmine as specific staining for Cn capsule (Lazcano et al., 1993), Russell's modification of Movat's pentachrome stain (Luna, 1992, and Verhoeff van Gieson (VVG) staining (Sheehan and Hrapchak, 1980). Movat is a pentachrome dye which stains mucin in alcian-blue, fibrous tissue in intense red, and elastic tissue in black. VVG stain is used to identify connective tissue, such as elastic fibers, which are stained in black, and collagen, which is stained in red. Also, organs were homogenized for tissue burden culture analysis in 10 ml PBS using the Stomacher 80 (Lab System, Fisher Scientific, Pittsburgh, Pa., USA) for 2 min at high speed. Serial dilutions were then plated onto YPD plates and incubated at 30° C. for 72 h and yeast colonies were counted and recorded as Colony Forming Unit (CFU) per organ. Data were recorded as the average ±standard deviation of Log₁₀ CFU/organ.

B. Results

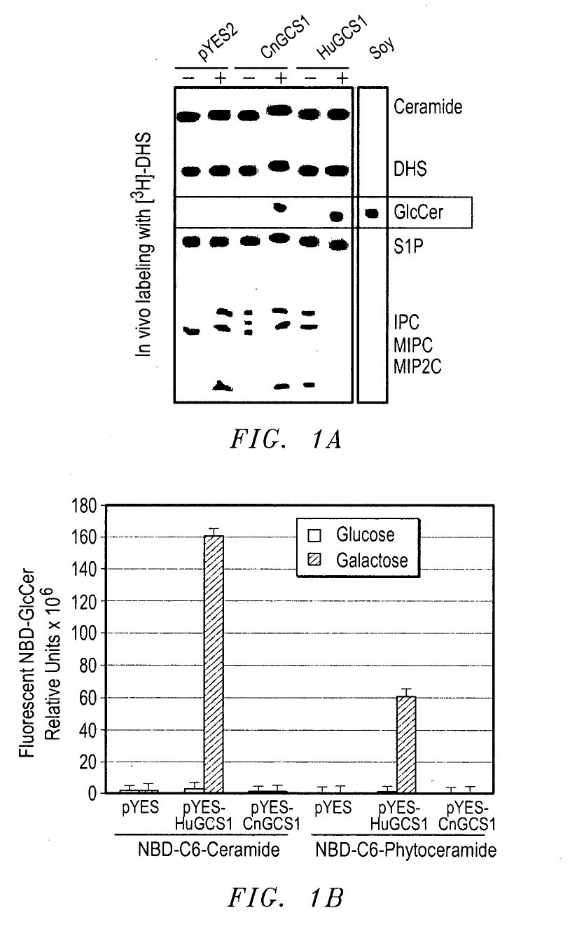

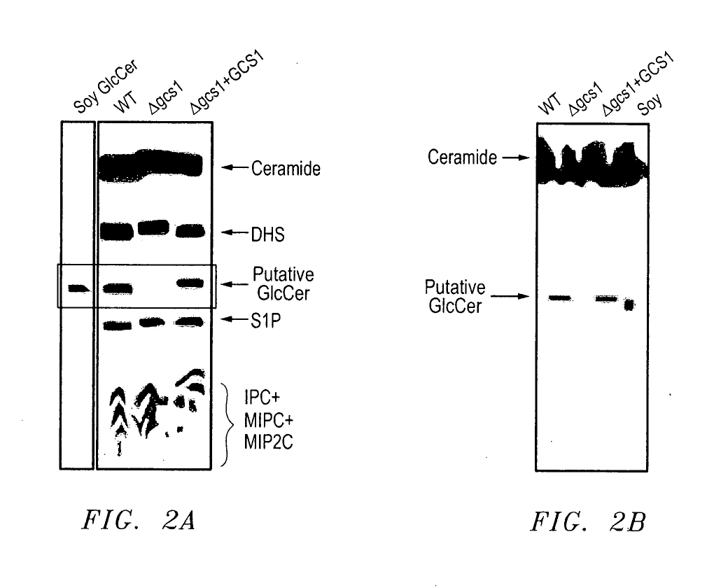

Cn GCS1 gene encodes for GCS activity. By sequence homology to the human GCS cDNA, a candidate Cn GCS1 cDNA was identified. To investigate the potential function of Cn GCS1 gene as GlcCer synthase, the effect of its expression on sphingolipid metabolism was studied in S. cerevisiae, which totally lacks endogenous GlcCer synthase activity. Human GCS gene was also expressed in a pYES vector as a positive control and S. cerevisiae carrying the pYES empty vector was used as a negative control. Yeast cells were labeled in vivo with tritiated dihydrosphingosine ([³H]-DHS) (FIG. 1A), which is a precursor of ceramide/phytoceramide (the substrate of GlcCer synthase) in mammalian and yeast cells. The results showed that, in galactose inducing condition, Cn Gcs1 produced a radiolabeled sphingolipid which migrated similarly to a soy GlcCer standard, and that this sphingolipid was absent in glucose repressing condition. As expected, expression of human GCS1 showed the formation of the putative GlcCer under inducing but not repressing condition (FIG. 1A). Cells carrying the empty vector lacked this sphingolipid under inducing or repressing conditions. These results suggest that Cn Gcs1 is functional when expressed in S. cerevisiae and that it may use endogenous ceramides as substrates.

Mammalian GlcCer synthase efficiently recognizes fluorescently labeled short chain ceramide analogs as substrates in cell free systems (in vitro) or in vivo. To study the substrate specificity of the Cn Gcs1, NBD-C6-ceramide and -phytoceramide were tested as substrates in vitro, using cell protein extracts of S. cerevisiae expressing Cn GCS1 or human GCS genes under repressing or inducing conditions. In contrast to what observed for human GlcCer synthase, Cn Gcs1 does not recognize NBD-C6-ceramides as substrates (FIG. 1B). The inability of Cn Gcs1 of using short chain fluorescent ceramide analogues was also confirmed by in vivo labeling using NBD-ceramides (data not shown). Altogether, these results show that Cn Gcs1 does not share the same substrate specificity of human GlcCer synthase.

Cn Gcs1 produces GlcCer. To examine whether Cn GCS1 gene encodes for GlcCer synthase, the Cn GCS1 gene was deleted in Cn var. grubii (WT), creating the Δgcs1 mutant, and subsequently reconstituted, generating the Δgcs1+GCS1 strain (FIGS. 6A-D). To verify that Cn Δgcs1 mutant lacks the putative GlcCer, in vivo labeling using [³H]-DHS in WT, Δgcs1 and Δgcs1+GCS1 strains was performed. As illustrated in FIG. 2A, a radiolabeled compound running similarly to the soy GlcCer standard was clearly lost in the Δgcs1 strain, whereas it was completely restored in the reconstituted strain. Use of NBD-ceramides in Cn for in vitro or in vivo labeling did not result in the formation of the corresponding identifiable sphingolipid in the tested strains (data not shown), in agreement with the findings that Cn Gcs1 expressed in S. cerevesiae was unable to recognize NBD-ceramide analogs as substrates.

To demonstrate that the missing sphingolipid in the Δgcs1 is GlcCer, we purified and concentrated the putative sphingolipid from Cn strains, and the extracted lipids were analyzed by high performance thin layer chromatography (HPTLC), electrospray tandem mass spectrometry, and Nuclear Magnetic Resonance (NMR). The analysis of the purified lipid extracts onto the HPTLC plate stained with orcinol clearly showed a band of a sugar-containing lipid migrating in correspondence of the GlcCer standard from Soy. This band was absent in the Δgcs1 strain and reconstituted in the Δgcs1+GCS1 strain (FIG. 2B).

The structure of the GlcCer was confirmed by syringe infusion of the glycosphingolipid into an API 3000 triple quadrupole mass spectrometer. Neutral loss scans of m/z 180 (loss of hexose headgroup) were performed as well as precursor ion scans for the types of sphingoid bases commonly found in fungi. A major peak in Cn WT was observed at m/z 756.7, which corresponds to a (M+H)⁺ for a monohexosylceramide with a lipid backbone comprised of 37 carbons, 2 double bonds, and an additional hydroxyl (FIG. 2C). Fragmentation of this ion yielded a product ion of m/z 276.4, which identifies the lipid backbone as having an 18-carbon sphingadienine base with one additional methyl group (for a total of 19 carbons) plus an amide-linked hydroxy-C18:0 fatty acid. This is consistent with the backbone structure of a GlcCer that has been previously isolated from this organism (9-methyl-4,8-sphingadienine in amide linkage to 2-hydroxyoctadecanoic acid) (Rodrigues et al., 2000). Precursor ion scans for m/z 276.4 also revealed lower abundance species that probably reflect GlcCer with a non-hydroxy-18-carbon fatty acid (at m/z 740.7) and a hydroxy-16-carbon fatty acid (at m/z 728.7). Precursor scans in the Δgcs1 mutant revealed minor peaks also present in the WT spectrum, whereas none of these GlcCers were detected (FIG. 2C). Importantly, the reconstituted strain (Δgcs1+GCS1) displayed a strong m/z 756.7 ion for the major GlcCer of the WT cells; interestingly, the minor 18:0 or hydroxy 16:0 species were not detected.

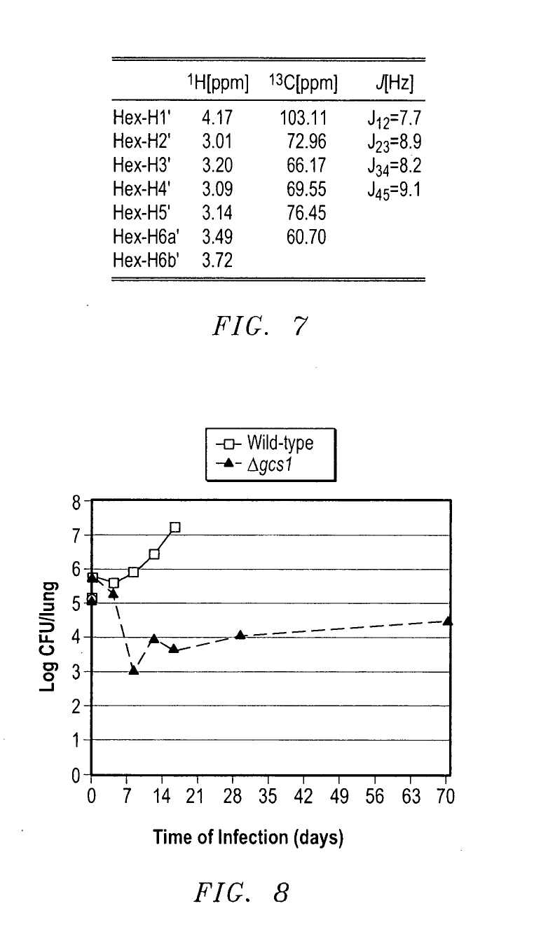

Since no standards are available for these compounds, LC mobility could not definitely identify the sugar head group. Thus, two-dimensional nuclear magnetic resonance (NMR) spectroscopy was applied to uniquely define the structure of the hexose moiety of the purified sphingolipid (WT), as shown in FIG. 2D. The NMR characterization of the hexose commenced with the assignment of the glycoside protons in the ¹H-NMR spectrum of the monohexosylceramide (FIG. 7). The unambiguous identification of the H1′ anomeric proton was straightforward due to its unique chemical shift (¹H: d 4.17 ppm; ¹³C: d 103.11 ppm) and doublet multiplicity (³J_{H1′,H2′}=7.7 Hz). A combination of two-dimensional ¹H, ¹H-(double-quantum filtered) COrrelation SpectroscopY (COSY) and -TOtal Correlated SpectroscopY (TOCSY) spectra clearly revealed the complete hexose proton spin system which was supported by the ¹³C Heteronuclear Single Quantum Correlation (HSQC) data (FIG. 2D). The stereochemistry of the hexose could be unambiguously determined from the consideration of the first four vicinal ¹³J-coupling constants. The large ¹³J_{HH}-coupling constants observed (>7 Hz) indicate antiperiplanar orientations of the vicinal hexapyranose H1′/H2′, H2′/H3′ (³J_{H2′,H3′}=8.9 Hz), H3′/H4′ (³J_{H3′,H4′}=8.2 Hz), and H4′/H5′ (³J_{H4′,H5′}=9.1 Hz) ring protons, respectively (FIG. 7). The analysis of 1D-NOE difference experiments corroborated the previous b-glycopyranosyl assignment. NOE's were observed from H1′ to H3′ and H5′; saturation at H2′ produced a strong NOE at H4′. Thus, based on these results and on the NMR analysis of GlcCer species from other pathogenic fungi (Toledo et al., 1999; Toledo et al., 2000), the inventors can conclude that glucose is attached to the ceramide backbone of the sphingolipid produced by Cn Gcs1 enzyme.

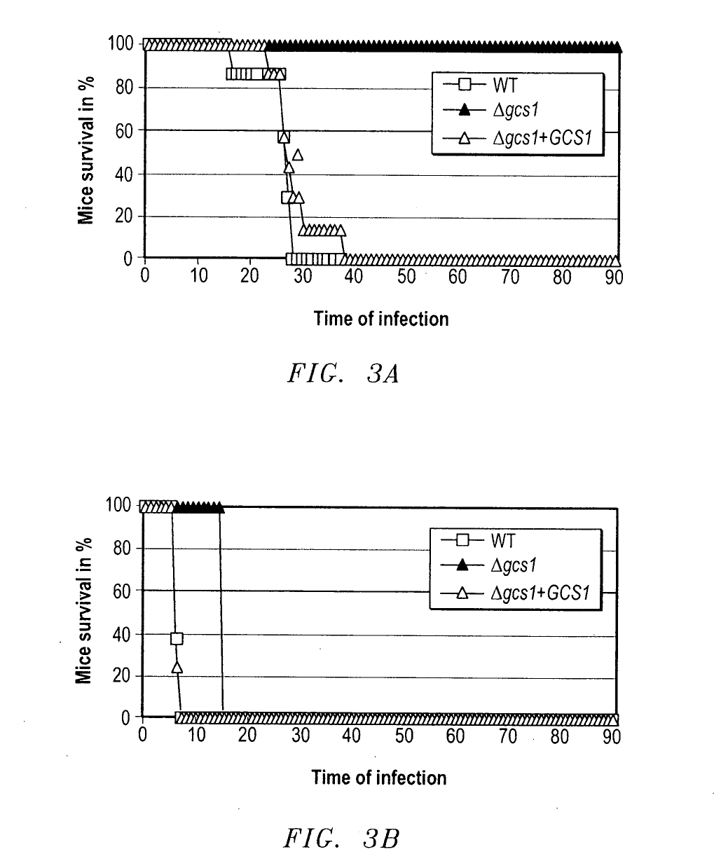

Role of Gcs1 on pathogenicity of C. neoformans. To establish the role of GCS1 on pathogenicity of Cn, the inventors performed virulence studies using a murine animal model (CBA/J) of cryptococcosis. The average survival of mice infected intranasally with WT or Δgcs1+GCS1 strains showed an average survival of 24.6±3.9 days and 27.3±4.8 days respectively (FIG. 3A) (P<0.0001) whereas mice infected with Cn Δgcs1 were all alive after 90 days of observation. At the 90th day, even though the 11 mice infected with Cn Δgcs1 mutant showed no clinical signs of meningo-encephalitis, they were sacrificed, and lungs and brains were removed and analyzed for colony forming units and histology. Yeast cells were absent in 8 out of 11 brains. Among the 3 positive brains, one showed ˜6×10⁵, one 500 and one 28,000 cells per organ. Among the 11 lungs, 6 lungs were used for fungal tissue burden and an average of 5-10×10³ yeast cells/lung were found, which corresponds to a ˜100-fold reduction of the initial inoculum. These results were confirmed by a study aiming to determine the lung fungal burden up to 70 days post infection with wild-type or Δgcs1 strain (FIG. 8). These findings suggest that Gcs1 plays a key role in pathogenicity of Cn and growth of yeast cells in the lung.

To examine whether the lung environment would have a role in protecting the host against the dissemination of Δgcs1 strain, the inventors challenged CBA/J mice intravenously with WT, Δgcs1, or Δgcs1+GCS1 strain and monitored mice survival. Mice infected with WT or Δgcs1+GCS1 strain showed a similar average survival rate of 6.3±0.5 days and 6.2±0.4 days respectively (FIG. 3B). Mice infected with Δgcs1 mutant were apparently healthy until 13-15 days of infection when they rapidly showed severe clinical signs of meningo-encephalitis, such as intense tremor, rigidity, lack of balance, and inability to coordinate movements. On the 15^{th} day, mice were sacrificed because they were unable to reach food or water. Brains, lungs, kidneys, spleens, and livers were removed and assayed for tissue burden culture and histology. Analysis of colony forming units (CFU) of brains showed an average number of 8.4±0.06 (Log₁₀ CFU±standard deviation) yeast cells, which was significantly higher (P<0.01) than CFU in other organs (5.5±0.5 in lung, 5.6±0.1 in kidney, 5.3±0.2 in spleen, and 5.6±0.1 in liver). These results clearly suggest that when CBA/J mice are challenged intravenously, they cannot efficiently contain Δgcs1 cells from disseminating throughout the body.

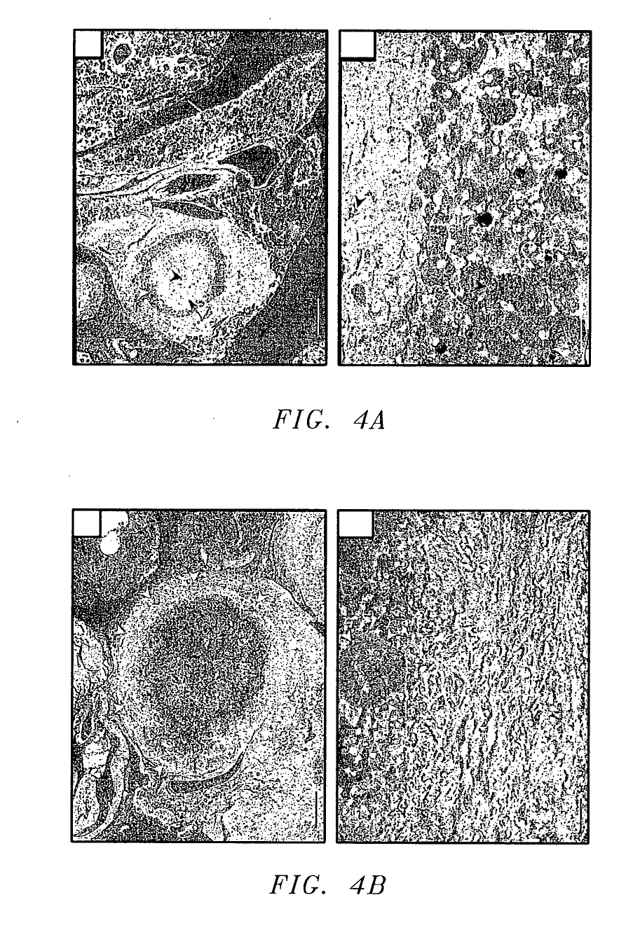

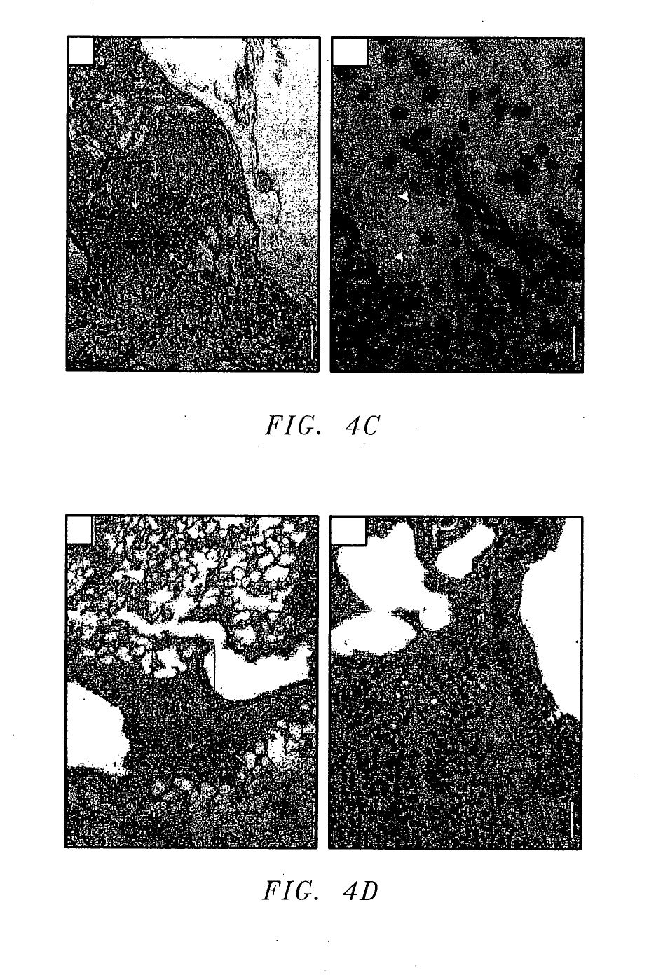

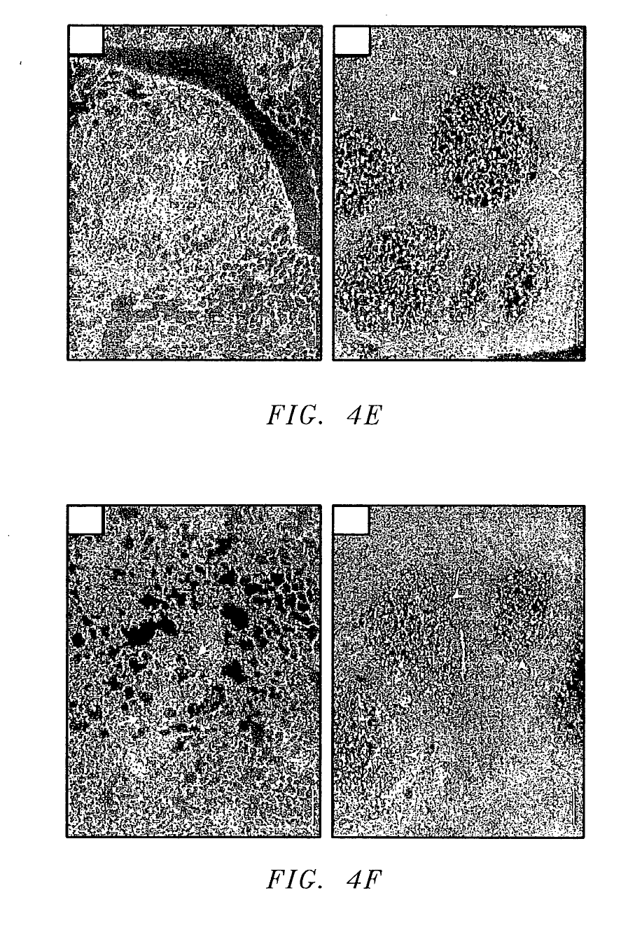

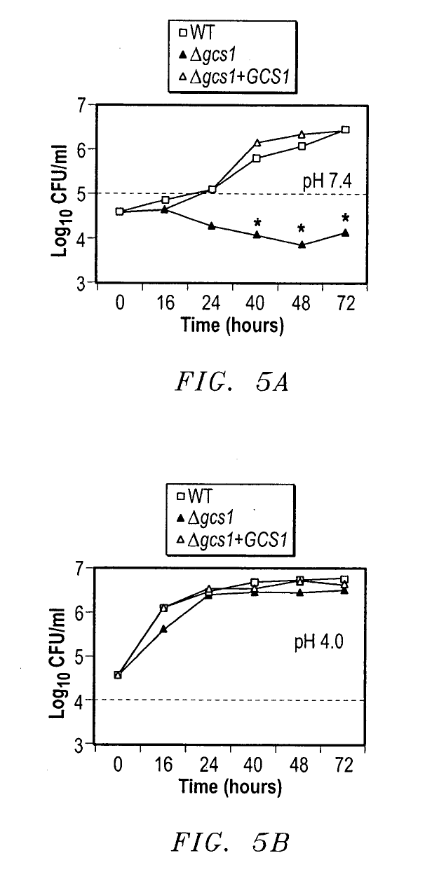

Inflammatory response against Cn Δgcs1 strain. To examine the host inflammatory response against Cn Δgcs1, 5 lungs and 5 brains from CBA/J mice challenged intranasally and 4 lungs and 4 brains of CBA/J mice challenged intravenously with Cn Δgcs1 were analyzed for histology. FIGS. 4A-4D shows representative fields from 4 different lungs recovered from mice infected intranasally. FIGS. 4EL and 4FL show a representative field of two lungs whereas FIGS. 4EB and 4FB show a representative field of two brains recovered from mice infected intravenously.

The lung of mice challenged intranasally showed a strong granulomatous response consisting in the formation of extensive spheric nodules in which Δgcs1 cells were mainly trapped in the center with abundant necrotic tissue and numerous neutrophils (FIGS. 4A and 4B). The necrotic tissue was surrounded by a ring of foamy macrophages and giant cells loaded with yeast cells and/or cellular debris or “ghosts” of yeast cells, which appeared to be degenerated (FIG. 4A1). The ring of macrophages was then surrounded by lymphocytes, fibroblasts and fibrotic tissue (FIG. 4A), in which a significant deposition of collagen could be observed (FIGS. 4B and 4B1). These nodules ranged from 500-2500 μm in diameter and were confined in the lung with no invasion into the mediastinal area. In other cases, smaller nodules were observed, such as those illustrated in FIGS. 4C and 4D, in which few Cn cells were engulfed in macrophages surrounded by other macrophages containing “ghosts” of Cn (FIG. 4C1) cells. Few lymphocytic interstitial infiltrations were localized in their proximity (FIGS. 4C-F). Other portions of the lung appeared to be normal. These structures were present only in lungs infected with Cn Δgcs1 with different degree of organization, whereas the nodules were not found in lungs of mice infected with Cn wild-type strain during the course of the infection (data not shown). All brains selected from mice infected intranasally with Δgcs1 and analyzed by histology showed no sign of yeast cells.

Histology analysis was also performed on lungs, brains and other organs of mice infected intravenously. In the lung, Δgcs1 cells are contained within small spheric nodule-like structure (FIGS. 4EL and 4FL) similar to those illustrated in FIGS. 4C and 4D. In the brain, Cn cells are almost exclusively localized within brain abscesses of different size, which are not surrounded by any host inflammatory response (FIGS. 4EB and 4FB). Numerous abscesses containing Δgcs1 cells were also found in the kidney (data not shown). In the liver, the Δgcs1 cells were found in very small periportal lesions with no host cellular infiltrates, whereas in the spleen the Δgcs1 strain was surrounded by a granulomatous response invading the white pulp and characterized by the presence of yeast cells surrounded by macrophages, lymphocytes, neutrophils and few magakaryocites (data not shown). These results suggest that when Cn Δgcs1 cells are introduced intranasally, the host responds with the organization of a strong granulomatous response, in which yeast cells are confined by macrophages, lymphocytes, fibroblasts, fibrotic tissue and collagen deposition, with the almost total containment of the infection within the lung tissue. In contrast, when yeast cells are introduced intravenously Cn Δgcs1 cells do disseminate to the brain (and other organs), eventually killing the animals.

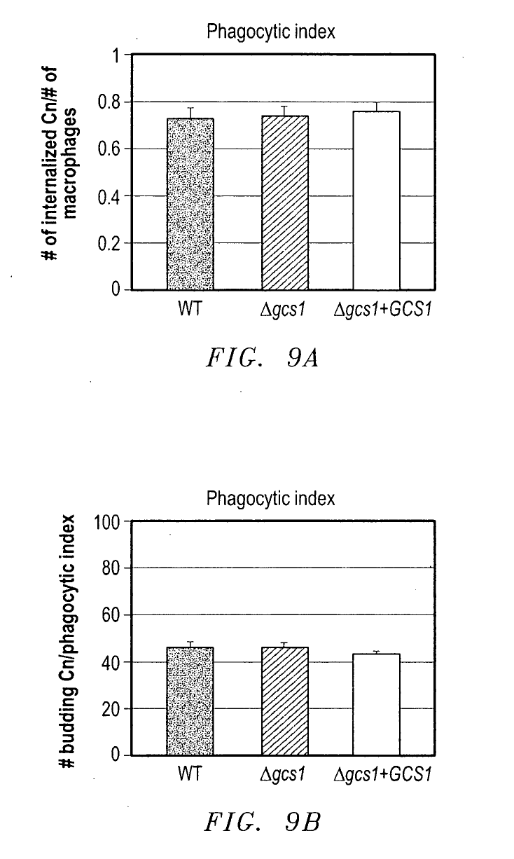

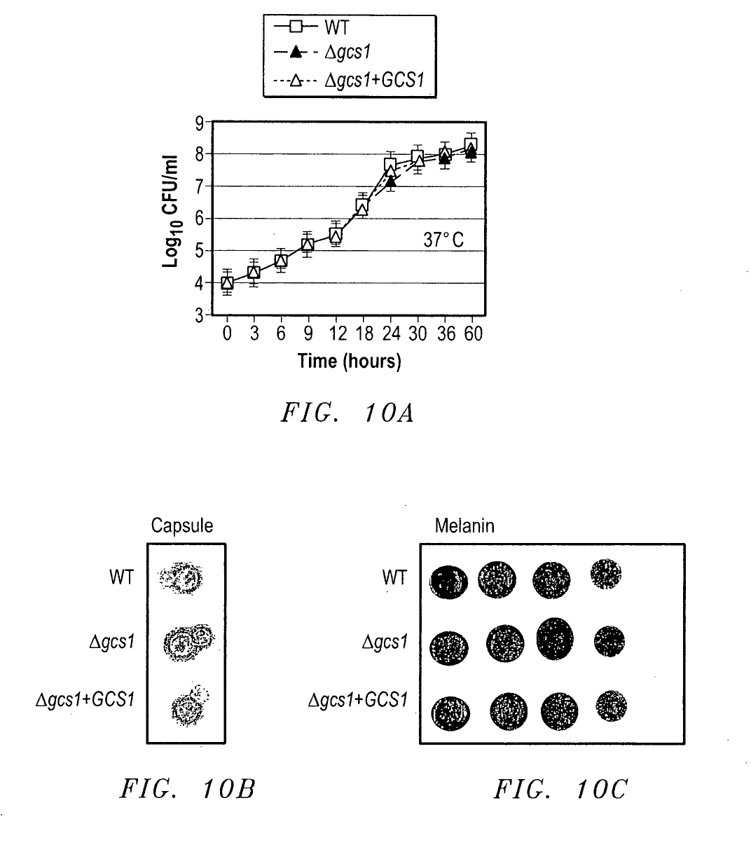

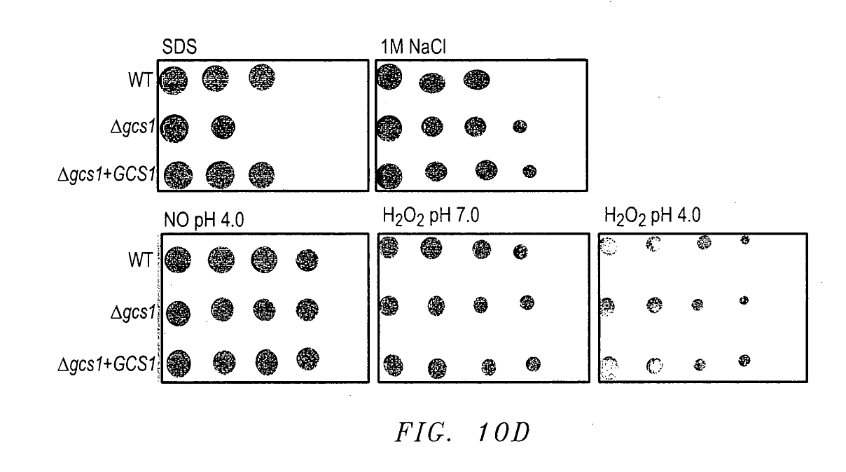

Effect of Gcs1 on intracellular and extracellular growth of Cn. In the host, Cn proliferates intracellularly, within the acidic environment of the phagolysosome, and extracellularly, an environment characterized by alkaline/neutral pH and high CO₂ concentration. Thus, the inventors investigated the effect of the loss of GlcCer on growth of Cn intracellularly and in vitro acidic and alkaline environments. For intracellular growth, Cn WT, Δgcs1, and Δgcs1+GCS1 strains were incubated with J774.16 macrophage-like cells and the number of buds of internalized cells per phagocytic index was measured. As indicated in the FIG. 9, the inventors found no differences in intracellular growth among the tested strains, suggesting that GlcCer has no effect on fungal growth once Cn is internalized within macrophages.

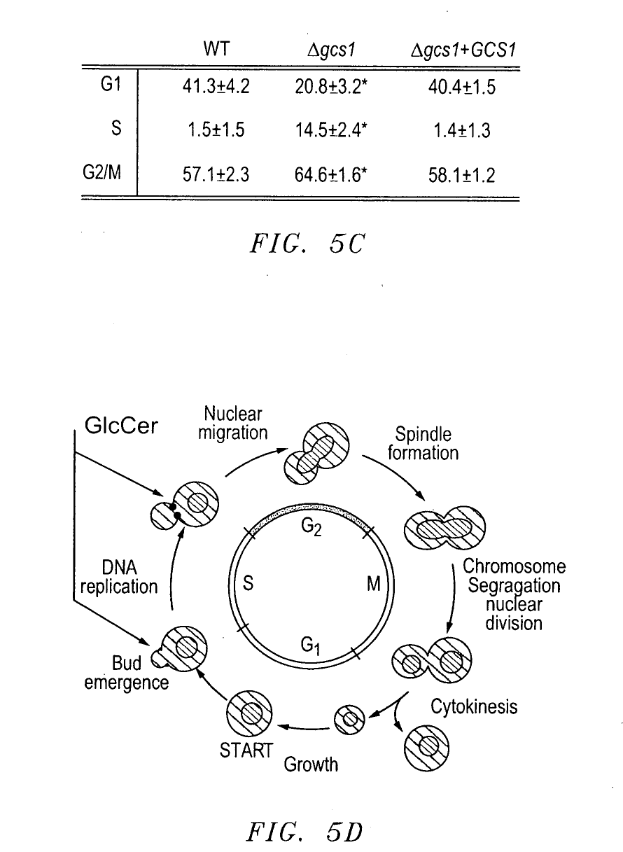

To evaluate the in vitro cell culture growth, we performed growth curves of Cn WT, Δgcs1, and Δgcs1+GCS1 strains incubated in 5% CO₂ at neutral/alkaline or acidic pH. As shown in FIG. 6A, the Δgcs1 mutant was unable to grow at neutral/alkaline pH in the presence of 5% CO₂ (FIG. 5A) whereas growth was observed at acidic pH (FIG. 5B). This difference in growth was not observed in presence of atmospheric CO₂ concentration (˜0.04%) at either pH 7.4 or 4.0 (data not shown). Importantly, the growth defect at 5% CO₂ and neutral pH is reversible because when yeast cells are switched to fresh acidic medium, cell growth is completely restored (data not shown), suggesting that growth of Δgcs1 cells is arrested at alkaline pH and 5% CO₂.

To examine which phases of the Cn cell cycle would be affected by lack of GlcCer, we performed flow cytometry analysis of Cn cells incubated for 24 h and 36 h at alkaline pH and 5% CO₂. The inventors found that a significantly greater percentage of Δgcs1 cells are locked in S and G2/M phases compared to the WT or Δgcs1+GCS1 cells at both 24 (FIG. 5C) and 36 h (data not shown), suggesting that GlcCer is important for exiting from the S and G2/M phases of the cell cycle

C. Discussion

In the present study, the role of Cn GlcCer synthase in virulence of this pathogenic fungus was investigated. The results show that Cn GlcCer synthase is an essential factor in determining the success of the fungal infection by regulating survival of Cn during the initial colonization of the lung. In addition, the inventors found that GlcCer synthase guarantees survival of Cn in the lung extracellular space by ensuring cell cycle progression at neutral/alkaline pH and physiologic CO₂ concentration (Table 1).

TABLE 1
Pathobiological features of Cn Δgcs1 mutant strain
In vitro

| | |
|---|---|
| Capule size | Similar to wild-type |
| Melanin production | Similar to wild-type |
| Growth at 37° C., ambient CO2 | Similar to wild-type |
| Growth in 0.05% SDS | Slightly decreased |
| Growth in 1 M NaCl | Similar to wild-type |
| Growth in 1 mM NO | Similar to wild-type |
| Growth in 5 mM H2O2 | Similar to wild-type |
| Growth at 37° C., 5% CO2, pH 4.0 | Similar to wild-type |
| Growth at 37° C., 5% CO2, pH 7.4 | Arrested |

In vivo

| | |
|---|---|
| Phagocytic index | Similar to wild-type |
| Growth within macrophages | Similar to wild-type |
| Growth in the lung | Arrested (if mice infected |
| | intranasally) |
| Lung inflammation | Granuloma-like structure |
| | with collagen deposition |
| | (if mice infected intranasally) |
| Dissemination to the brain | Significantly decreased |
| | (if mice infected intranasally) |
| Mice survival | 100% at 90 days |
| | (if mice infected intranasally) |

The dramatic protection against pathogenicity imparted by loss of GlcCer synthase underlines the importance of the extracellular component of fungal cells during Cn infection. Cn infects the host through inhalation and when arrives in the alveoli it encounters two environments in which normally grows: the extracellular space, characterized by a neutral/alkaline pH and a physiologic CO₂ concentration of ˜5.0% (compared to 0.04% present in the air (Villee, 1957); and the intracellular environment of the phagolysosome of alveolar macrophages characterized by an acidic pH. Our results suggest that GlcCer is critical for Cn to grow in the extracellular (alkaline) and not in the intracellular (acidic) environment of the lung. Since during the infection most Cn cells are found in the host extracellular space (Levitz, 2001) the overall effect of the loss of GlcCer on Cn pathogenicity is significant. Indeed, when the Δgcs1 strain is introduced intranasally, the overall growth of yeast cells in the alveoli is arrested (FIG. 8) and, even if the mutant can grow intracellularly (FIG. 9), the host responds with the formation of a well organized granulomas (FIGS. 4A-F) which will efficiently contain Δgcs1 cells in the lung. This allows the host to survive the cryptococcal infection without any evident clinical sign of disease (FIG. 3A).

When the Δgcs1 strain is introduced intravenously, mice survived longer than those infected with WT or reconstituted strain (FIG. 3B). In the blood, Δgcs1 also finds a hostile environment because the pH is ˜7.4 and CO₂ concentration is ˜5% (PCO₂˜40 mm Hg). Thus, growth of Δgcs1 cells in the bloodstream would be arrested until yeast cells are either internalized by phagocytic cells or they leave the bloodstream and invade the organs in which they form abscesses, such as those found in the brain (FIGS. 4EB and 4FB) or in the kidney (data not shown). In both the intracellular environment and in abscesses the pH is characteristically acidic and growth of Δgcs1 can resume. Indeed, it was interesting to note that in the brain upon intravenous challenge of Δgcs1, yeast cells were almost exclusively contained within abscesses (FIGS. 4EB and 4FB), whereas wild-type cells, in addition to being present in abscesses, were also present in surrounding tissue (data not shown). Thus, the inventors hypothesize that once the acidic abscess is formed, Δgcs1 cells promptly restore growth, and the consequent destruction of brain tissue would eventually manifest abruptly with clinical sign of meningoencephalitis. Thus, the animal infected intravenously with Δgcs1 would eventually die although it would take longer than mice infected with the control strains due to the transient growth arrest that yeast cells will undergo in the bloodstream upon injection.

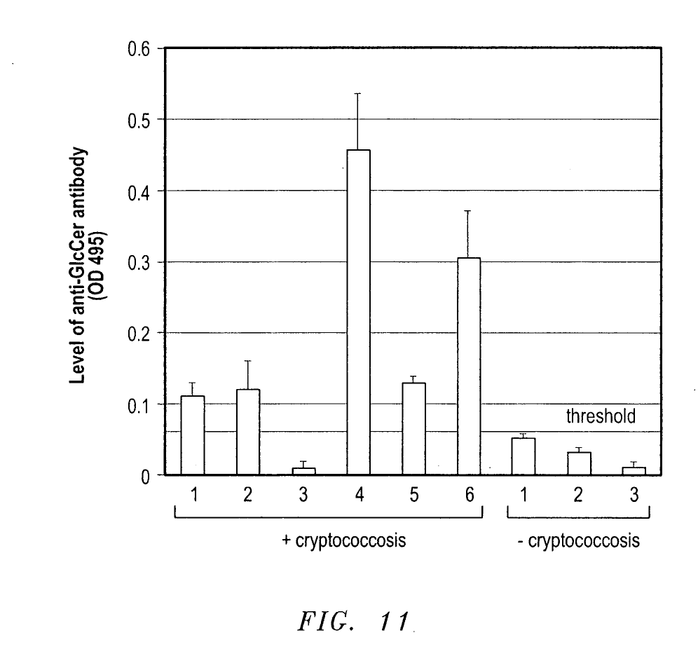

The results from these studies identify GlcCer as a novel virulence factor for Cn because loss of this glycosphingolipid does not affect other fungal characteristics, such as capsule production, melanin formation or growth at ambient CO₂ and 37° C. (FIGS. 10A-D), known to be required to cause disease (Casadevall and Perfect, 1998). Interestingly, a greater sensitivity of Δgcs1 mutant to SDS was observed (FIGS. 10A-D) suggesting an alteration of cell wall integrity. This hypothesis is supported by previous observations in which the density of GlcCer at the cell surface correlates with the cell wall thickness of Cn (Rodrigues et al., 2000). However, the difference in cell growth of Δgcs1 in the presence of SDS compared to control strains is not so dramatic to account for the significant loss of virulence of the mutant. Instead, the growth of Δgcs1 mutant is arrested in environments characterized by alkaline pH and 5% CO₂.

In budding yeasts, such as Cn, the S and G2 phases of the cell cycle are characterized by the bud formation and maturation (FIG. 5D). Interestingly, previous studies have shown that GlcCer is localized at the budding site of Cn and addition of human antibodies against Cn GlcCer to in vitro Cn culture inhibits cell budding and fungal cell growth (Rodrigues et al., 2000). Our studies showed that loss of GlcCer significantly prolongs the S and the G2/M phases in alkaline environments therefore suggesting a mechanism whereby GlcCer regulates fungal cell growth by allowing yeast cells to complete the cell cycle.

The role of GlcCer synthase in alkaline tolerance is not restricted to Cn as recent findings showed that a Kluyveromyces lactis mutant defective in GlcCer can not grow at pH 8.5 (Saito et al., 2006). On the other hand, the molecular mechanism by which GlcCer regulates alkaline tolerance at high but not low CO₂ is not known. Studies have proposed that fungal cells sense different concentrations of CO₂ and, in conditions in which CO₂ is elevated (e.g., 5%), fungi respond by up-regulating virulence factors. For instance, CO₂ stimulates capsule formation in Cn (Granger et al., 1985) and the transformation of yeast cells to filamentous forms in Candida albicans through adenylyl cyclase (Klengel et al., 2005). In other pathogenic fungi, such as Coccidioides immitis, CO₂ promotes the formation of endosporulating spherules (Klotz et al., 1984), which is a process required for virulence. These studies suggest the existence of a conserved signaling pathway that regulates fungal virulence through the sensing of CO₂. Cn senses CO₂ through two β-class carbonic anhydrases (Can 1 and Can2), which have been recently identified (Mogensen et al., 2006; Bahn et al., 2005), but whether GlcCer regulates signaling events mediated by CO₂-carbonic anhydrases at different pHs awaits further investigations.

In conclusion, these studies show that GlcCer plays a key role in the pathogenicity of Cn through a mechanism that ensures the transition through the cell cycle of yeast cells in alkaline environments with physiological concentration of CO₂. Since in cases of cryptococcosis extracellular organisms predominate, the function of GlcCer becomes clinically relevant. Interestingly, Gcs1 and GlcCer are found in a variety of pathogenic fungi and because a significant biochemical difference between human and Cn Gcs1 exists, compounds that would specifically inhibit the fungal enzyme might represent a novel class of antifungal agents against fungal infections (Levery et al., 2002; Thevissen et al., 2004). Furthermore, since antibodies against the fungal GlcCer inhibit Cn growth in vitro and they do not interact with the human GlcCer (Toledo et al., 2001), they may represent a new therapeutic approach to control infections due by fungal microorganisms producing GlcCer.

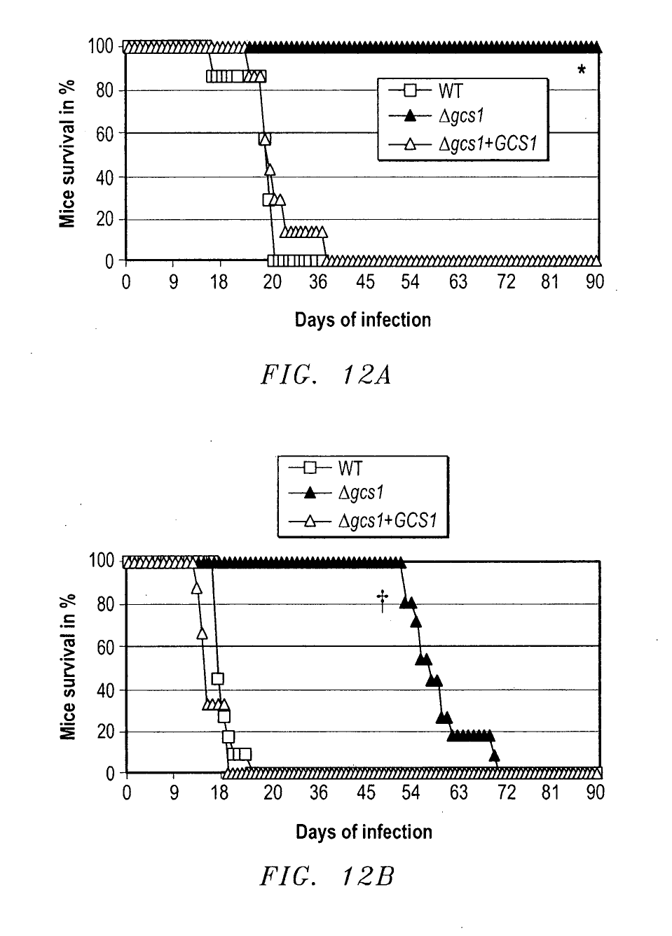

The presence of antibody anti-GlcCer in sera of HIV patients with cryptococcosis and not in normal uninfected subjects was determined by an ELISA assay (FIG. 11). These results show that this detection method is specific and sensitive enough to propose large scale studies. Interestingly, the inventors found a significant difference in the level of anti-GlcCer antibody among positive patients. Very interestingly, sample (patient) #3 showed no antibody against GlcCer (FIG. 11). Follow-up investigations revealed that sample #3 is from a patient affected by a cryptococcal disease confined in the lung and the mediastinal lymph nodes with no dissemination of the fungus in the bloodstream or the brain. The infectious disease doctor treating patient #3 felt that by the time the sample was collected, the cryptococcosis had been adequately treated. This suggests that either the antibodies against GlcCer disappear when the disease resolves or are not produced if the fungus does not leave the lung and migrates into the bloodstream.

### Example 2

Although other investigators have shown the presence of antibody against glucosylceramide in sera of patients affected by cryptococcosis, it is not known at which stage of the cryptococcal disease these antibodies are produced. Thus, the have tested inventors are currently testing human clinical samples for the presence of anti-glucosylceramide antibody using an ELISA assay in various patient populations (e.g., those affected by cryptococcosis with and without fungal meningo-encephalitis).

The presence of anti-glucosylceramide (anti-GlcCer) antibodies in human fluids of patients with cryptococcosis will be determined and evaluated. In preliminary studies, the inventors showed that some patients affected with cryptococcosis produced antibody anti-GlcCer, but it was not known whether the level of this production would be constant during the infection nor was the host immune status at the time of blood sampling known. Thus, an epistasis analysis of the determination of anti-GlcCer production in relation to the host immune status and the severity of the cryptococcal disease will be performed. This will be achieved by collecting serial samples (sera, cerebrospinal fluids, bronco-alveolar ravages) from the same and different patients and assaying for the level of anti-GlcCer antibody. All clinical information regarding the underlying disease and all parameters/data regarding the fungal disease will also be collected.

The determination of anti-GlcCer antibody will be performed by an indirect ELISA. Briefly, serial concentrations of purified GlcCer will be absorbed in wells of an ELISA plate 2 hr at room temperature. After washing with PBST, the plate will be incubated with human samples at different dilutions in phosphate buffered saline-tween 20 (PBST) for 1 hr at room temperature. The plate will be washed with PBST, and anti human antibody conjugated to horseradish peroxidase will be added at 1:1,000 dilution as a secondary. After the incubation with 3,3,5,5-tetramethylbenzidine for 5-20 minutes, plates were read at 450 nm using a spectrophotometer. Assay optimization might include mouse anti-GlcCer antibody (MEST-2) as a positive control.

Thus, if GlcCer is required to leave the lung, we hypothesize that its production would increase when the fungus would invade the bloodstream. As a consequence, it is at this early stage (higher production of GlcCer) that the host would respond with a higher production of antibody against GlcCer. Clearly, the identification of this early stage is clinically significant because an early antifungal treatment could block the development of the neurological disease, which would otherwise be diagnosed only when clinical signs of meningo-encephalitis would occur.

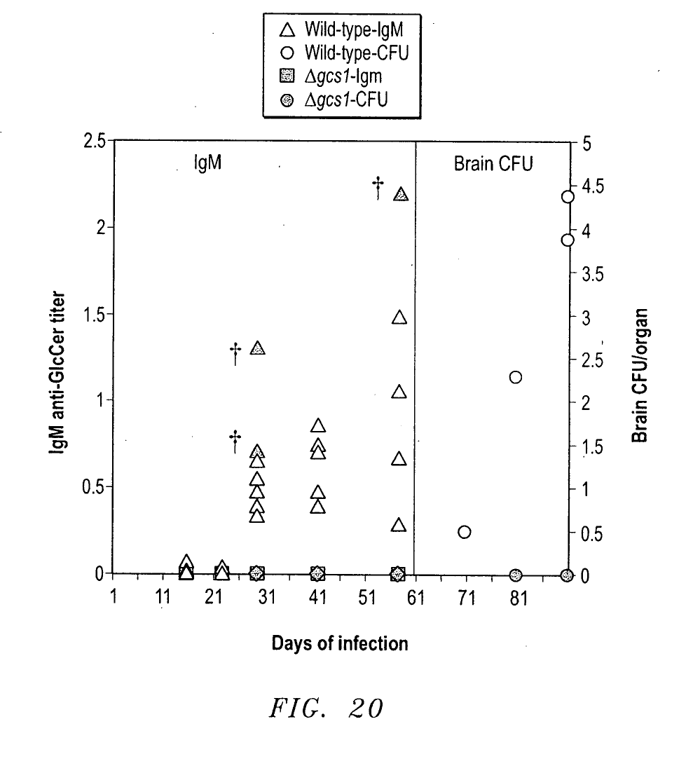

The hypothesis that the presence of anti-GlcCer antibody can be detected in sera before the dissemination of the fungus is strongly supported by the studies conducted in the inventors' laboratory aimed at the production of monoclonal antibody against GlcCer. To produce monoclonal antibody against GlcCer, they infected mice intranasally with a low dose of Cn wild-type cells that do produce GlcCer. As a control, we intranasally infected mice with Δgcs1 mutant cells that do not produce GlcCer. At the indicated time points after the infection, they collected the blood from the safenous vein (without killing the mouse) from both wild-type and mutant infected mice and measured anti-GlcCer antibody in the sera. As illustrated in FIG. 20, mice infected with Cn wild-type cells do have IgM antibody against glucosylceramide at different titer, most likely due to the inevitable variability among different mice. Noteworthly, mice with lower titer at day 29 of the infection will ultimately increase their titer on a later day. Perhaps, the increased production of antibody is due to the increased production of the GlcCer, which, as the inventors showed in previous studies (Rittershaus et al., 2006), it is an essential molecule for fungal replication of Cn during the infection. As expected, sera collected from mice infected with Δgcs1 were negative for antibody against GlcCer. Some of these mice were sacrificed at the illustrated time points and lung, brain, kidney, spleen and liver were removed. The spleen was fused to a myeloma cell line to produce a hybridoma for antibody production. The other organs were tested for the presence of Cn cells. Cn cells were only found in the lung and not in liver, kidney or brain. These results strongly suggest that the presence of IgM anti-GlcCer in sera is detected prior to the dissemination of Cn cells from the lung to other organs. At later time points, days 70, 80 and 90, the inventors sacrificed the remaining mice and examined their organs for CFU. As illustrated in FIG. 20, Cn wild-type cells do eventually disseminate to the brain causing meningoencephalitis in the same mouse in which we detected anti-GlcCer at early time points. As expected, Δgcs1 cells did not disseminate to the brain as also previously observed (Rittershaus et al., 2006). These studies suggest that antibody against GlcCer can potentially predict that fungal dissemination from the lung to the brain may soon occur.

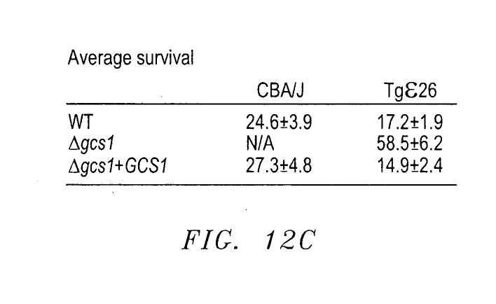

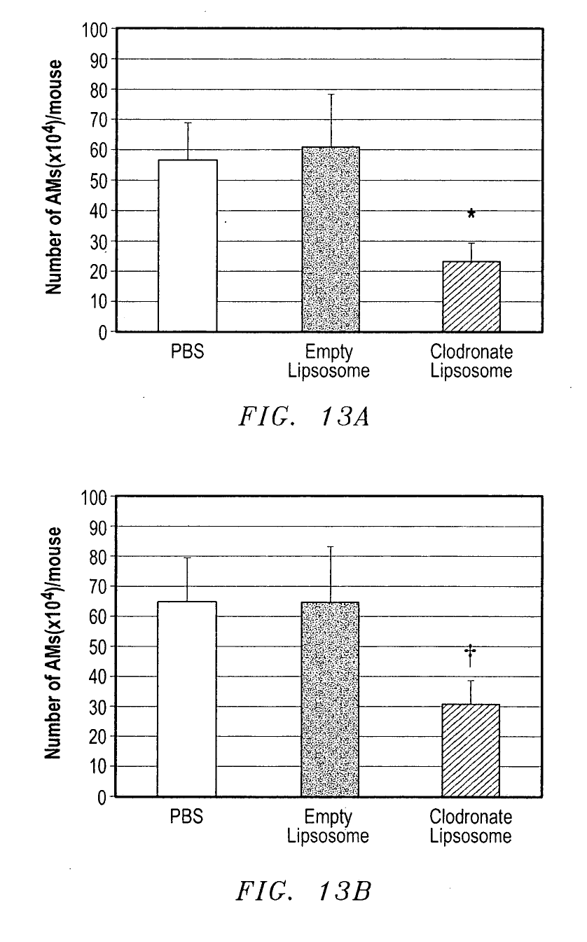

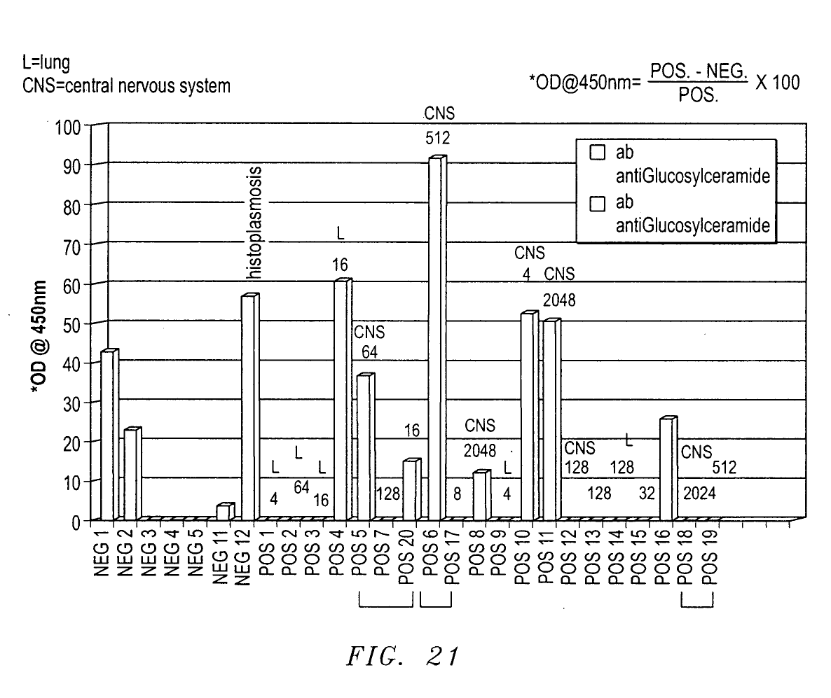

To test whether they were able to detect anti-GlcCer antibody using an ELISA assay in sera of human subjects, the inventors performed a pilot experiment by testing serum samples stored in the Clinical Microbiology Laboratory at the Medical University of South Carolina. Samples were given to us with the information of whether the Cn capsule antigen polysaccharide was present (+) or absent (−) from serum (see also FIG. 12). As discussed above, this test detects fungal particles of Cn in the serum and its presence in the bloodstream is an indication that the fungus is most likely present in the bloodstream and, thus, that Cn dissemination already occurred. As illustrated in FIG. 21, they did find different titers of IgM anti-GlcCer antibody in sera of patients positive for Cn polysaccharide antigen but, interestingly, the antibody titer does not correlate with the antigen titer. For instance, patient POS6 shows an antigen titer of 1:512 and an antibody titer of 90, whereas patient POS8 shows an antigen titer of 1:2048 but a much lower antibody titer of ˜12 (FIG. 21). In addition, the antibody titer does not correlate with the involvement of the central nervous system (CNS) because patients with meningoencephalitis (CNS) do have different antibody titers in their serum (FIG. 21). In addition, the inventors also identified patients with positive antigen but negative anti-GlcCer antibody. These results suggest that once Cn has already disseminated into the bloodstream, the anti-GlcCer antibody is most likely not a good marker for diagnosis. On the other hand, perhaps the test can be used as a prognostic marker. These results were not correlated with the severity of the meningoencephalitis, the outcome of cryptococcosis or/and the efficacy of the antifungal therapy because these informations were not available. Some patients with lung involvement and low Cn antigen titer (POS1, POS2, POS3, POS7, POS9 and POS14) had no anti-GlcCer antibodies. It is possible indeed that the absence or presence of low levels of said antibodies may indicate an effective antifungal treatment.

Most importantly, however, is the finding that patients with negative Cn antigen do have anti-GlcCer antibody present in their serum. This is quite intriguing because it may indicate that these patients might have an active fungal infection in the lung. Of significant interest is patient NEG12 showing high titer of anti-GlcCer antibody. This patient was diagnosed with pulmonary histoplasmosis without any involvement of other organs and tissues. These results suggest that the present invention can be potentially used for the diagnosis of fungal infections other than cryptococcosis.

These studies were performed in a very limited sample size and with fragmented clinical informations. The retrospective study illustrated in FIG. 21 was done just to prove that we are able to detect anti-GlcCer in human samples. The inventors are currently submitting a collaborative grant in which they propose to enroll all transplant patients admitted at the Medical University of South Carolina and to follow them throughout a 2 years period for the presence of anti-GlcCer antibodies in their blood and for the development of fungal infection(s). This prospective study will most likely yield more useful information as to whether this test can predict the development of invasive fungal infection(s).

TABLE 1
Anti-GlcCer antibody titer in patients in which cryptococcal
polysaccharide antigen was negative (NEG 1, 2, 4 and 5) or positive
(POS 3, 4, 5, 6, 10, 11 and 21)

| SAMPLES | $OD @ 450 nm = \frac{POS - NEG}{{POS}^{*}} ⨯ 100$ | DILUTION FACTOR |
|---|---|---|
| NEG1 | 50 | 1:1536 |
| NEG2 | 21 | 1:6144 |
| NEG4 | 0 | 1:192 |
| NEG5 | 0 | 1:192 |
| POS3 | 0 | 1:192 |
| POS4 | 42 | 1:6144 |
| POS5 | 45 | 1:3072 |
| POS6 | 85 | 1:24576 |
| POS10 | 81 | 1:6144 |
| POS11 | 58 | 1:3072 |
| POS21 | 0 | 1:192 |

*Titer (OD 450 nm) is compared to the dilution factor, which is the dilution of the serum made to test for the antibody.

### Example 3

A. Materials and Methods

Strains, media and reagents. C. neoformans var. grubii serotype A H99-derived strains Δgcs1 and Δgcs1+GCS1 are previously described in great detail (Rittershaus et al., 2006). H99 (wild-type) and H99-derived strains Δgcs1 and Δgcs1+GCS1 were grown in yeast extract/peptone/2% dextrose (YPD) media from Difco. YPD plates used were supplemented with chloramphenicol (100 μg/ml) and ampicillin (100 μg/ml). All reagents used are from Sigma Chemical Co. (St. Louis, Mo.) unless otherwise specified.

Animal studies. Four to six-week-old Tgε26 mice (Wang et al., 1994; Wang et al., 1997), available in our Animal Core Facility, Medical University of South Carolina, Charleston, S.C., were used for this study. Mice were anesthetized with an intraperitoneal injection of 60 μl xylaxine/ketamine mixture containing 5 mg xylazine and 95 mg ketamine per kilogram of body weight. Wild-type and mutant strains of Cn were grown in YPD media for 24 hrs at 30° C. The yeast cells were harvested and washed three times by centrifugation at 3000 rpm for 10 minutes and resuspended in sterile PBS (pH=7.4) at a concentration of 2.5×10⁷ cells/ml. Mice were infected intranasally with 20 μl containing 5×10⁵ wild-type, Δgcs1 or Δgcs1+GCS1 cells. Mice were fed ad libitum and monitored by twice-daily inspections. Mice that appeared moribund, in pain or with clinical signs of meningoencephalitis were sacrificed using CO₂ inhalation followed by cervical dislocation. All animal procedures were approved by the Medical University of South Carolina Institutional Animal Care and Use Committee and followed the guidelines of the American Veterinary Medical Association. Survival data from the mice experiment were analyzed by Kluskal-Wallis test. A p value of less than 0.05 was considered to be significant.

Alveolar macrophage depletion. Liposomes containing clodronate (dichloromethylenediphosphonic acid disodium salt) and empty liposomes used as control, were prepared as described previously (Van Rooijen, 1994). Briefly, 8 mg cholesterol (Avanti Polar Lipids) and 86 mg L-alpha phosphatidylcholine (Avanti Polar Lipids) were dissolved in chloroform, which was then slowly evaporated. The thin, filmy layer was resuspended in 10 ml of PBS or 0.6M clodronate. The mixture was exposed to N₂ gas and incubated 2 hours at room temperature with gentle shaking. The mixture was then sonicated and incubated another 2 hours to allow liposome swelling. The solution was centrifuged at 10,000 g for 15 minutes and the liposomes containing clodronate were collected and washed twice with sterile PBS. The liposomes were kept at 4° C. under N₂ until use for a maximum of 2 weeks.

Alveolar macrophage depletion was evaluated by intranasally introducing 60 μl of PBS, empty liposome or liposomes containing clodronate into the mouse and performing bronchoalveolar lavage at 48 hours or 6 days. Bronchoalveolar lavage was done by inserting a stub adapter in the trachea of the mouse and rinsing the alveolar spaces 10 times using 0.5 ml PBS (pH=7.4) for each wash. The collected fluid was spun down and alveolar macrophages were counted. Lavage samples were taken from two mice per time point per group. Experimental groups were divided into five mice each receiving PBS (control group), empty liposome (control group) or liposome encapsulated clodronate administered mice. Two days before infection with yeast, the mice were anesthesized and given 60 μl of PBS, empty liposomes or clodronate-encapsulated liposomes. After 48 hrs, mice were infected with the yeast cells as described above. Mice continued to receive PBS, liposome or clodronate treatment weekly.

Tissue fungal burden. Lungs, brain, spleen and the kidney were removed aseptically from each mouse on days 4, 8 and 12 for wild-type infected mice and days 16, 26, 36, 50, 64 and 70 for Δgcs1 infected mice (although only those treated with clodronate were able to survive the last two time points). Organs were homogenized in sterile PBS, using the Stomacher 80 (Lab system, Fisher Scientific, Pittsburgh, Pa., USA) for 2 minutes. Serial dilutions were then plated onto YPD plates and colony-forming units were counted and recorded. Statistical analysis was performed using Student's t test, and a p value of less than 0.05 was considered to be significant.

Histological analyses. Organs were harvested and fixed for 48 hours in 37% formaldehyde, paraffin-embedded, sectioned and stained with movat, mucicarmine, or hematoxylin and eosin.

B. Results

Virulence of Cn Δgcs1 in mouse models. To determine the role of GlcCer in the pathogenicity of Cn we performed virulence studies using two murine animal models of cryptococcosis: an immunocompetent model CBA/J (as previously reported in (Rittershaus et al., 2006)) and an immunodeficient model Tgε26, in which both T and NK cells are deficient. The average survival of CBA/J mice infected with WT or Δgcs1+GCS1 was 24.6±3.9 and 27.3±4.8 days respectively, whereas mice infected with Cn Δgcs1 were all alive at 90 days (P<0.0001; FIG. 12A). The average survival of Tgε26 mice infected with WT or Δgcs1+GCS1 was 17.2±1.9 and 14.9±2.4 days, respectively, whereas the average survival of mice infected with Cn Δgcs1 mutant was 58.5±6.2 days (P<0.001, FIG. 12B). These results suggest that Tgε26 mice lack the immune mechanism(s) that contain the disease namely the T and NK cells which play a key role in the containment of the Δgcs1 mutant within lung granulomas.

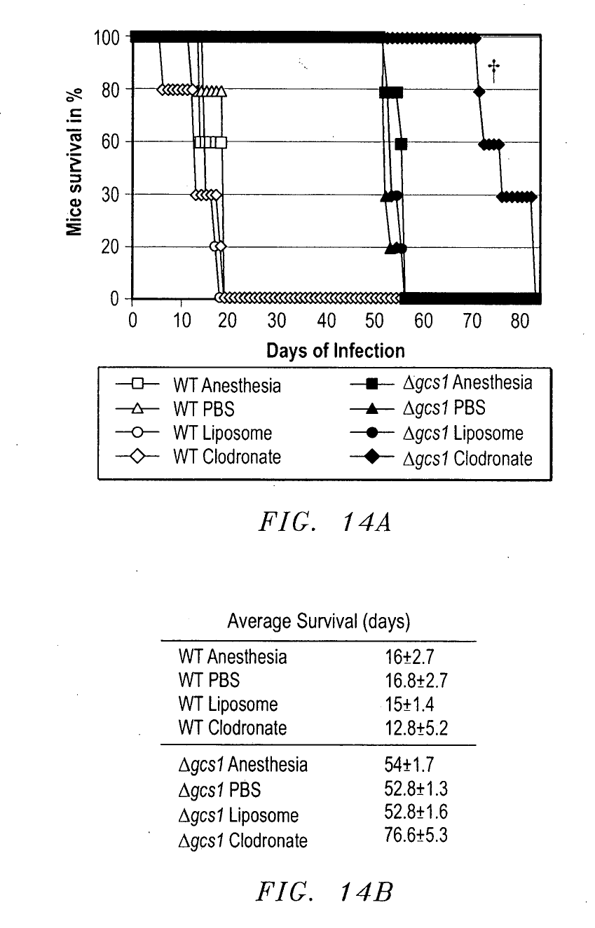

Pharmacological depletion of alveolar macrophage. Liposomes containing clodronate were prepared as described in the materials and methods. Sixty microliters of liposome suspension of clodronate was intranasally injected into each mouse. Two control groups were used: one group received 60 μl of empty liposomes and one received 60 μl of PBS. After 48 hours or 7 days from the injection, mice were sacrificed, the bronco alveolar lavage (BAL) fluid was collected and viability of alveolar macrophages assessed microscopically. After 48 hours, clodronate-treated mice showed a significant decrease (58%) in number of alveolar macrophages (AMs) compared to PBS- or empty liposometreated mice (P<0.05) (FIG. 2). After 7 days, clodronate-treated mice showed a 52% reduction of AMs compared to control-treated mice (P<0.05) (FIG. 13). These results showed that the administration of a single dose of liposome-contained clodronate is sufficient to decrease AMs by 50% within 48 hrs and this decrease is maintained for at least for 7 days. These results are consistent with previously reported observations (Shao, 2005) and with the fact that complete regeneration of AMs in mice requires 18 days.

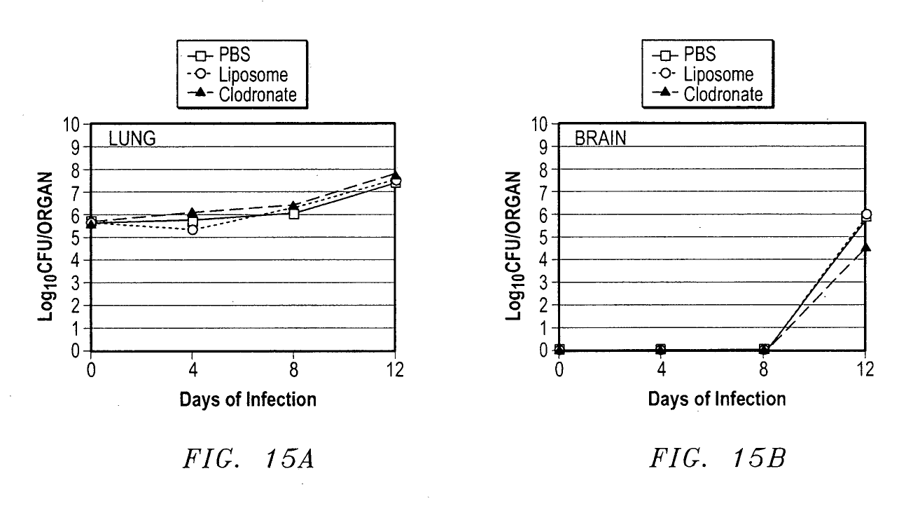

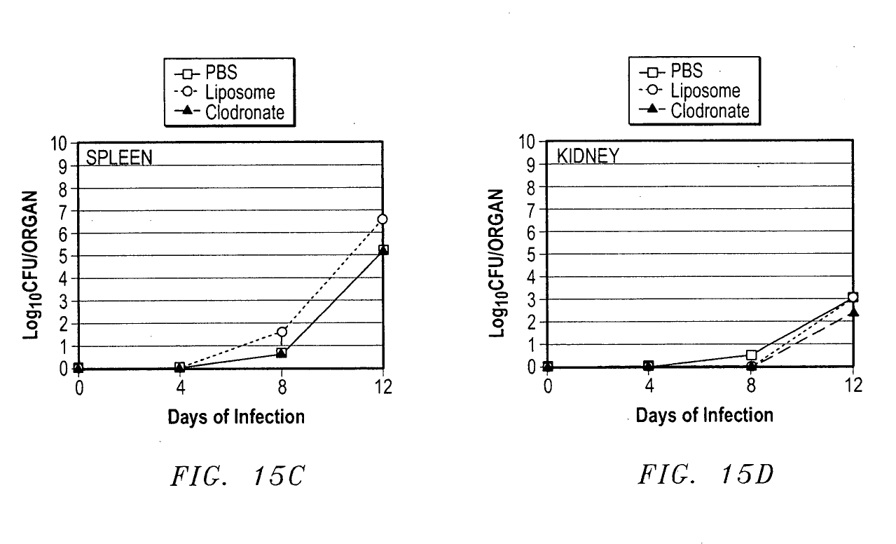

Effect of macrophage depletion on survival of Cn-infected mice. After 48 hours of clodronate or control treatment, mice received an intranasal injection of Cn wild-type or Δgcs1 mutant, as described in materials and methods. In addition, since mice would have to be subjected to repeated anesthesia because of the weekly administration of liposomes or PBS, an additional group of infected mice receiving only a weekly anesthesia without liposome or drug injection were included as a control for anesthesia. The average survival of Cn-infected mice receiving anesthesia alone, PBS control, empty liposome and clodronate was 16±2.7, 16.8±2.7, 15±1.4, 12.8±5.2 respectively (FIG. 14). Overall, clodronate-treated mice infected with Cn WT did not show a significant change in survival as compared to the control groups. On the other hand, the average survival of Δgcs1-infected mice receiving clodronate was significantly increased (76.6±5.3) compared to the average survivals observed in Δgcs1-infected mice receiving anesthesia alone, PBS control, or empty liposome (54±1.7, 52.8±1.3, and 52.8±1.6, respectively) (P<0.05) (FIG. 14). These results showed that Δgcs1-infected mice with fewer AM's survived significantly longer than mice having a normal number of alveolar macrophages, suggesting that these phagocytic cells may enhance the progression of disease by favoring the development and the dissemination of Δgcs1 cells in Tgε26 mice.

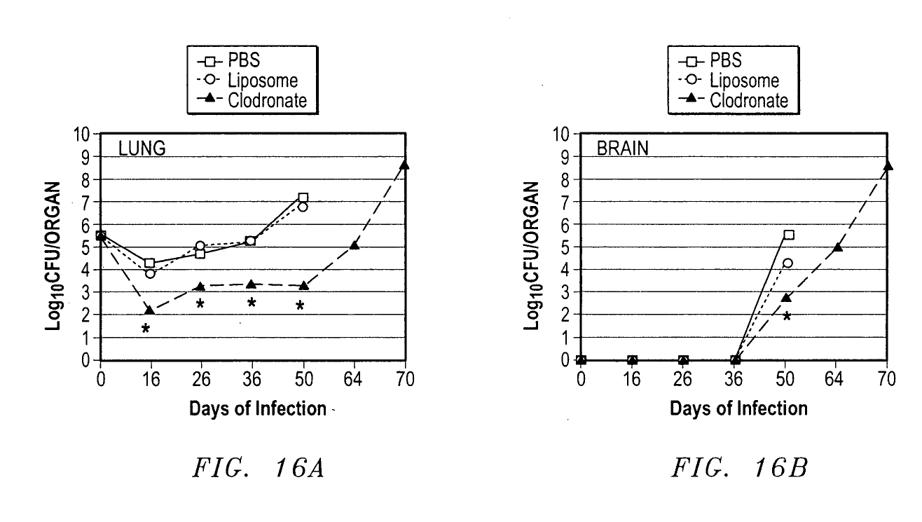

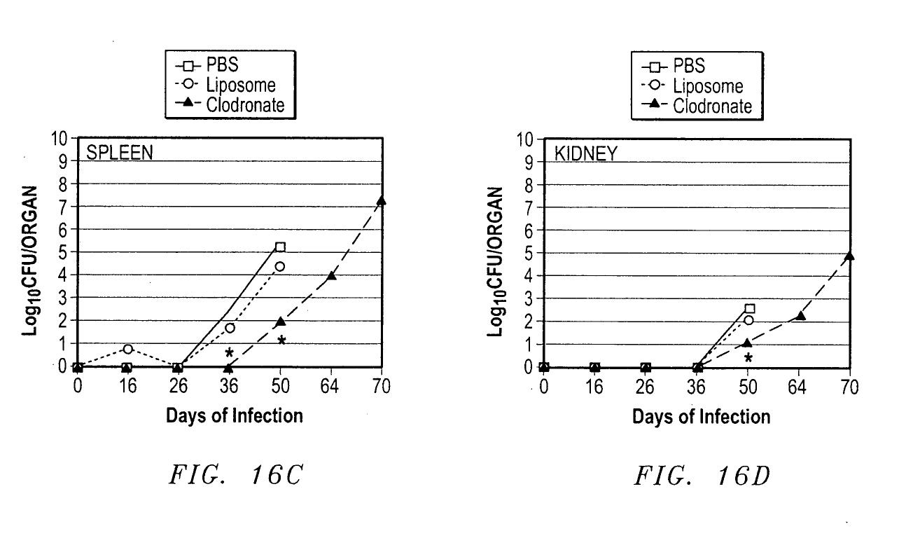

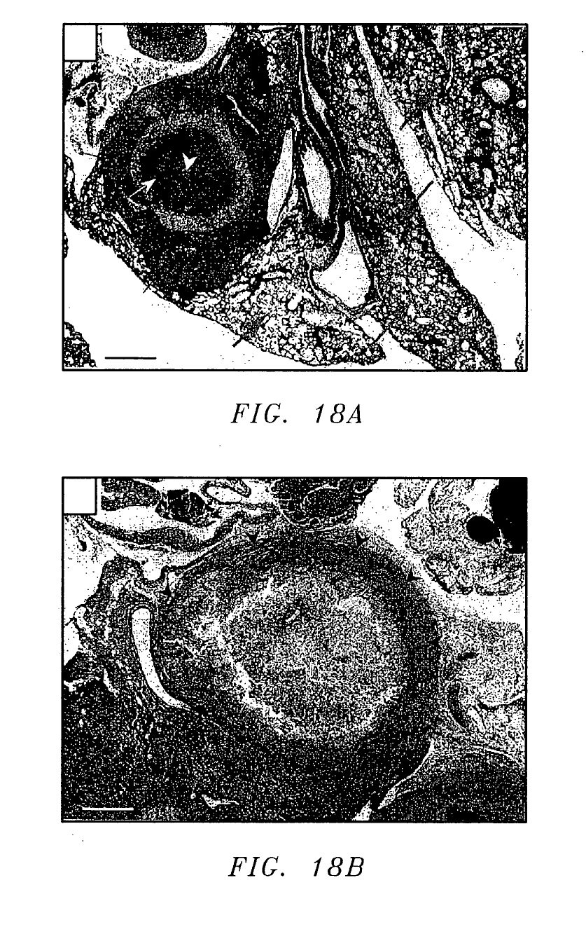

Tissue burden studies. To address the effect of macrophage depletion on the dissemination of Δgcs1 cells, we performed tissue burden studies of lung, brain, spleen, and kidney upon infection of Cn WT or Δgcs1 strain. No statistically significant differences were found in tissue colonization of Cn WT cells in all organs analyzed among the infected groups that received either PBS or empty- or clodronate-liposome (FIG. 15). Interestingly, mice infected with Δgcs1 mutant treated with clodronate showed a significantly lower number of fungal cells in all organs analyzed compared to control treated groups (FIGS. 16A-B) (P<0.05). Of interest, lung infected with the Δgcs1 mutant showed an early decrease in fungal cell number in all three experimental groups (FIG. 16A), a condition that was also observed in our previous study performed in immunocompetent CBA/J mice (FIG. 18) (Rittershaus et al., 2006). This decrease in fungal load is attributed to the death/clearance of fungal cells not able to growth in the lung extracellular environment. An interesting point is the dramatic decrease in the Δgcs1 cells in the macrophage-depleted Tgε26 lungs (FIG. 16A). It is remarkable that a decrease of alveolar macrophages by ˜50% would reduce the lung Δgcs1 fungal load >3000-fold (P<0.001). Eventually, the Δgcs1 cells do seem to grow in the lung although the significant delay is clearly observed in the macrophage-depleted mice. Similarly, in all other organs there seems to be a delay of 14 days in fungal growth in the clodronate-treated as compared to the control-treated mice. These results suggest that depletion of AMs significantly delay the fungal growth of Δgcs1 cells in the lungs and other organs of Tgε26 immunocompromised mice.

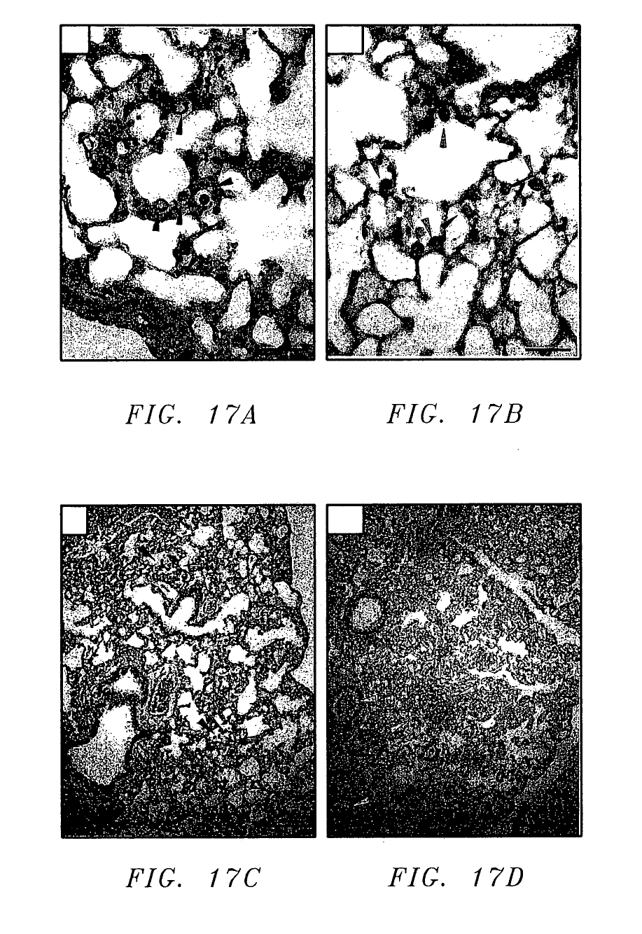

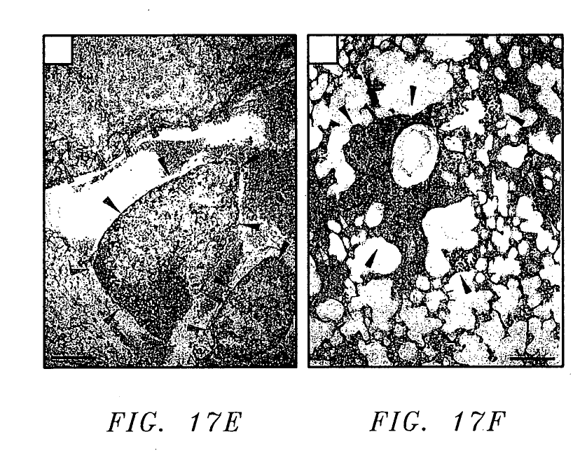

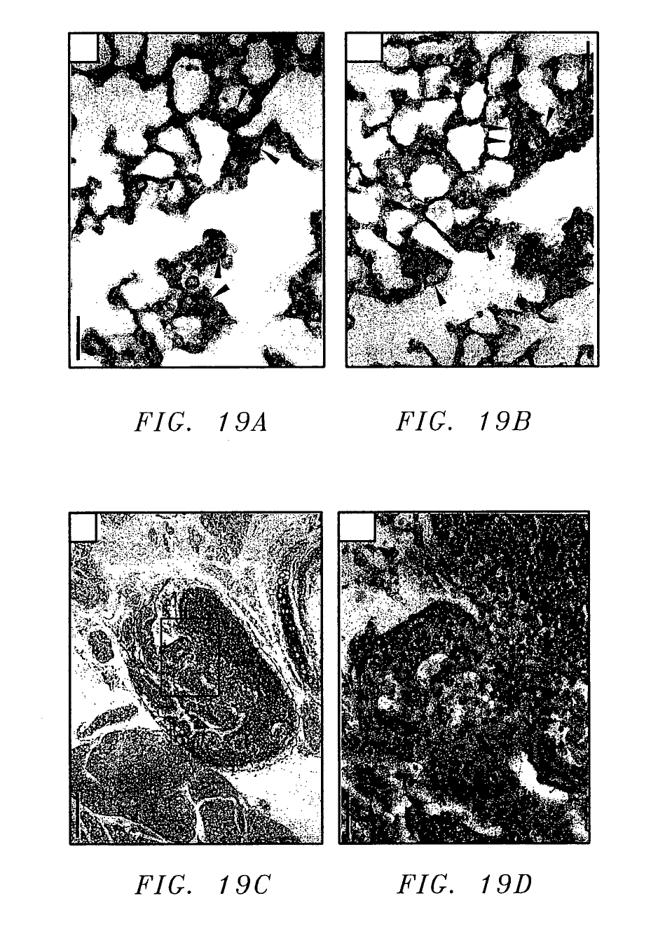

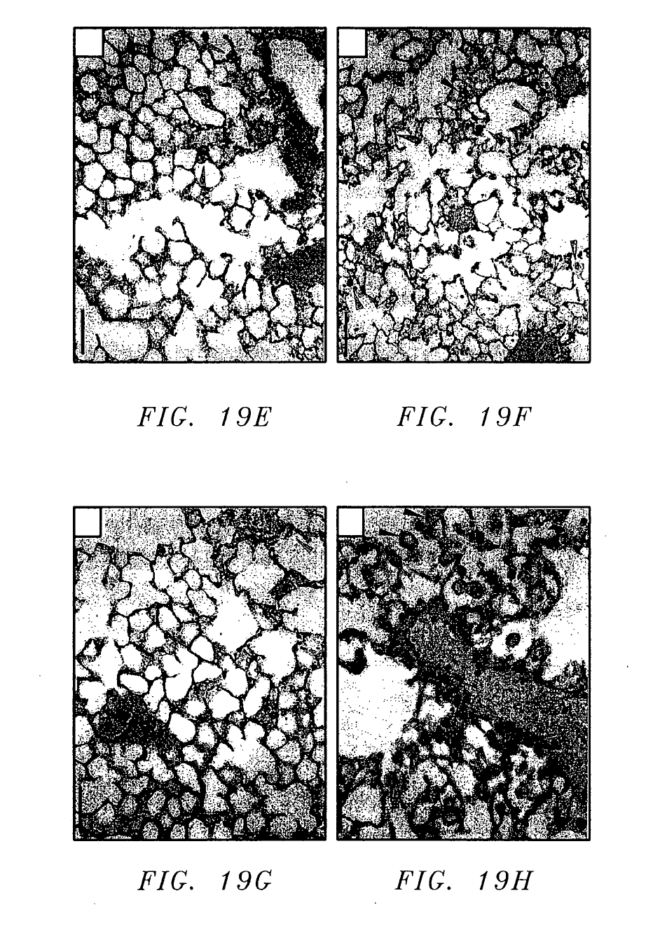

Histopathology. To gain further insights in the host inflammatory response against Cn Δgcs1 cells in mice in which macrophages have been deleted, we performed a histology analysis on lungs of Tgε26 mice infected with Δgcs1 strain and receiving empty liposome or liposome containing clodronate. In mice treated with empty liposome at 16 days of infection, Δgcs1 cells were found predominantly intracellularly and contained in the lung parenchyma (FIG. 17A and FIGS. 19A and 19B) or in lymph nodes (FIGS. 19C and 19D) with a moderate or absent host inflammatory response. As the infection progresses (36 day), the number of Δgcs1 cells is increased in mice treated with empty liposomes with a moderate infiltration of macrophages (FIG. 17C), and by 50 days of infection, Δgcs1 cells have significantly disrupted the lung structure and infiltrated the lymph nodes with destruction of the tissue (FIG. 17E). In contrast, lung of mice treated with clodronate showed few Δgcs1 cells in the lung parenchyma at day 16 (FIG. 17B and FIGS. 19E, 19F, 19G and 19H) and day 36 (FIG. 17D), and these fungal cells are mostly in the extracellular environment. Very few Δgcs1 cells were found intracellularly (FIGS. 19B, 19F and 19H). These lungs are characterized by the absence of an inflammatory response and by the lack of lymph node infiltration. Small granulocyte infiltrations are observed at days 36 and 70 of the infection but these were limited to few areas of the lung (FIG. 17F). It is remarkable that Δgcs1 cells are not found in the alveolar spaces but almost exclusively the lung parenchyma and mostly intracellular (FIG. 17F). These results showed that in Δgcs1-infected T and NK deficient mice depleted of AMs significantly decreased cryptococcal disease progression compared to T cell- and NK-deficient mice in which AMs were not depleted.

C. Discussion

Cn is a facultative intracellular pathogen known to survive and replicate within macrophages in vitro (Diamond and Bennett, 197a3; Feldmesser et al., 2001). Within non-activated macrophages, Cn replicates more rapidly than extracellularly; in immunocompromised individuals, Cn survives intracellularly where it can grow unthreatened by the compromised host cellular response. Thus, Cn may have developed regulatory mechanisms controlling pathogen invasion and dissemination based on its preference for the intra- or extracellular compartment, although it is unclear which component of the cryptococcal population, intra- vs. extracellular, most affects the disease progression (Diamond and Bennett, 1973b; Huffnagle et al., 1998; Chang et al., 1998; Levitz et al., 1999; Tucker and Casadevall, 2002). Fungal virulence or the hosts' immune response status may influence the choice of an intra- or extracellular lifestyle. For instance, under conditions of impaired cell-mediated immunity, intracellular fungi may proliferate uncontrolled due to lack of AM activation and killing. This hypothesis is supported by our studies and other reports suggesting that Cn disseminates within macrophages from the lung to the mediastinal area and to the brain when the host cellular response is impaired (Chretien et al., 2002; Luberto et al., 2003).

Very recent findings suggest that depletion of AM's in mice leads to a decrease of Cn proliferation in the lung at 3 and 14 days post-infection (Shao et al., 2005), supporting the hypothesis that Cn uses AMs to grow and replicate in the lung. On the other hand, upon phagocytosis, activated AMs can effectively kill Cn (reviewed in Del Poeta, 2004). In this respect, phagocytosis can be considered either an opportunity or an obstacle for Cn to produce disease, an aspect that is worth investigating to elucidate the importance of Cn-AMs interactions in cryptococcal disease development and progression. Thus, the identification of genes/factors of the pathogen or host that contribute to the intra- or extracellular preference will be useful for development of novel strategies for prevention and treatment of fungal infections.

In previous studies, the inventors identified that the sphingolipid pathway is a key regulator of the lifestyle of Cn. For instance, the production of glucosylceramide is essential for growth in environments characterized by an alkaline and neutral pH and 5% CO2, which are mainly found in the extracellular lung spaces (Rittershaus et al., 2006). If the host is immunocompetent and can respond with activation of AM and the formation of granuloma (FIGS. 18A and 18B), most of Δgcs1 cells will be contained in the lung. Lung containment allows the host to survive the cryptococcal infection without any evident clinical sign of disease. If the host is immunocompromised (e.g., lacking T and NK cells), it cannot build a lung granulomatous response (Casadevall and Perfect, 1998). Although the alveolar space of the immunodeficient mouse is hostile for growth of Δgcs1 cells, Cn can also grow inside AM's within the acidic environment of the phagolysosome despite the high concentration of reactive oxygen and nitrogen species (Rittershaus et al., 2006). Thus, once inhaled by T cell- and NK cell-immunodeficient mice, Δgcs1 cells may survive in the intracellular environment of macrophages, in which they are free to grow. This hypothesis is supported by the observation that although growth in vitro of Δgcs1 mutant in 5% CO2 at alkaline pH is arrested it can be restored if cells are switched to low pH (data not shown). Since dissemination of Cn may occur within the phagolysosome of AM's (Chretien et al., 2002; Luberto et al., 2003; Santangelo et al., 2004; Harrison and levitz, 2002 in which the pH is acidic, the inventors hypothesize that in the T cell- and NK cell-deficient mouse model Δgcs1 disseminates mainly within AM's eventually killing the host.

This hypothesis was explored by investigating the effect of the depletion of AMs in T cell- and NK cell-deficient mouse model on Cn infection by Δgcs1 mutant. When AM's are depleted the virulence of Δgcs1 mutant is decreased because the dissemination from the lung to other organs is significantly delayed and this is accompanied by the increase of survival. It is remarkable that although these Tgε26 mice lack T cells and NK cells and ˜60% of their total AM's, they survive infection by the Δgcs1 strain for an additional ˜18-20 days. Since Δgcs1 cells are unable to grow in the extracellular spaces, they are contained in the lung parenchyma, mostly within the remaining macrophages. Eventually, the Δgcs1 mutant finds conditions (e.g., within the remaining AMs or within other phagocytic cells such as dendritic cells and granulocytes), in which its growth is restored.

Interestingly, a recent study by Alvarez and Casadevall showed that Cn can exit the macrophage through an extrusion of the phagosome, while both the released Cn and macrophage cells survive and are able to replicate (Alvarez and Casadevall, 2006). When applied to the Δgcs1 mutant, the latter phenomenon will eventually lead to an increase of the fungal burden even in the presence of few AM's because one AM can serve as a haven for many Δgcs1 cells. The increased fungal burden will ultimately kill of the host. Thus, this study clearly shows that AM's exacerbate cryptococcosis when the host is severely immunodeficient. The hypothesis that Cn can be disseminated within macrophages is also supported by the decreased dissemination of Cn wild-type to the brain in AM-depleted mice (FIG. 15B). These results further corroborate previous studies (Chretien et al., 2002; Luberto et al., 2003; Shao et al., 2005; Shea et al., 2006) suggesting that macrophages can be used by Cn to disseminate to the brain.

The identification of GlcCer as a new virulence factor for Cn infection has prompted the inventors to engage in further studies of this molecule to clarify its role during cryptococcal disease. Being structurally different from mammalian GlcCer, they envision the possibility of targeting fungal GlcCer using monoclonal antibodies. Interestingly, antibody against GlcCer exerts an inhibitory effect on in vitro growth of fungal cells and enhances the antifungal activity of macrophages (Rodrigues et al., 2000; Nimrichter et al., 2004. The inventors believe that monoclonal antibodies not only can be used for prevention but also for treatment of fungal infections caused by microorganisms producing this glycosphingolipid.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### XI. REFERENCES

The following references, to the extent that they provide exemplary procedural or other details supplementary to those set forth herein, are specifically incorporated herein by reference.
U.S. Prov. Ser. 60/802,678
Alvarez and Casadevall, Curr. Biol., 16:2161-5, 2006.
Bahn et al. Curr. Biol., 15:2013-2020, 2005.
Bennett et al., Clin. Infect. Dis., 36:S123-127, 2003.
Boas et al., Chem. Phys. Lipids, 70:11-19, 1994.
Bowden et al., Clin. Infect. Dis., 35:359-366, 2002.
Bradford, Anal. Biochem., 72:248-254, 1976.
Calderone and Cihlar, Fungal Pathogenesis, 1-762, Basel, Marcel Dekker AG, NY, 2002.
Casadevall and Perfect, Cryptococcus neoformans, 381-405, ASM Press, Washington, D.C., 1998.
Centers for Disease Control and Prevention: Summary of notifiable Diseases, United States. MMWR Morb. Mortal Wkly Rep., 51:29-30, 2002.
Chang et al., J. Immunol. Methods, 211:51-63, 1998.
Chretien et al., J. Infect. Dis., 186:522-530, 2002.
Clearly et al., Am. J. Pharmacogenomics, 5:365-386, 2005.
Cox et al., J. Med. Vet. Mycol., 34:385-391, 1996.
Dabrowski, Two-Dimensional NMR Spectroscopy. Applications for Chemists and Biochemists, 8:757, VCH Publishers, Inc., NY, 1994.
Del Poeta, Eukaryotic Cell, 3:1067-1075, 2004.
Delaglio et al., J. Biomol. NMR, 6:277-293, 1995.
Diamond and Bennett, Infect. Immun., 7:231-6, 1973.
Diamond and Bennett, J. Infect. Dis., 127:694-697, 1973.
Feldmesser et al., Infect. Immun., 68:4225-4237, 2000.
Feldmesser et al., Trends Microbiol., 9:273-278, 2001.
Garber, Drugs, 61 (Suppl 1):1-12, 2001.
Granger et al., J. Clin. Invest., 76:508-516, 1985.
Groll et al., J. Infect., 33:23-32, 1996.
Gumbo et al., Pediatr. Infect. Dis. J., 21:54-56, 2002.
Hajjeh et al., J. Infect. Dis., 179:449-454, 1999.
Hakim et al., Aids, 14:1401-1407, 2000.
Hanada, Jpn. J. Infect. Dis., 58:131-148, 2005.
Harrison and Levitz, Cryptococcus neoformans and macrophages, Marcel Dekker, Inc., New York, 2002.
Herbrecht et al., N. Engl. J. Med., 347:408-415, 2002.
Heung et al., Infect. Immun., 74:28-39, 2006.
Heung et al., J. Biol. Chem., 279:21144-21153, 2004.
Heung et al., J. Biol. Chem., 280:28547-28555)
Heyderman et al., Clin. Infect. Dis., 26:284-289, 1998.
Hopfer and Amjadi, Fungal pathogenesis: principles and clinical applications, 649-665, Calderone and Cihlar (ed.), Marcel Dekker, Inc., NY, 2002.
Huffnagle et al., J. Immunol., 160:2393-400, 1998.
Husain et al., Emerg. Infect. Dis., 7:375-381, 2001.
Ichikawa et al., Proc. Natl. Acad. Sci. USA, 93:4638-4643, 1996.
Kaplan et al., Cancer, 39:2265-2274, 1977.
Klengel et al., Curr. Biol., 15:2021-2026, 2005.
Klotz et al., J. Infect. Dis., 150:127-134, 1984.
Koerner et al., Methods Enzymol., 138:38-59, 1987.
Kohno et al., Kansenshogaku zasshi, 68:1512-1517, 1994.
Lazcano et al., Mod. Pathol., 6:80-84, 1993.
Lee et al., Lab. Invest., 73:871-879, 1995.
Leipelt et al., Biochem. Soc. Trans., 28:751-752, 2000.
Leipelt et al., J. Biol. Chem., 276:33621-33629, 2001.
Levery et al., FEBS Lett., 525:59-64, 2002.
Levitz et al., Infect. Immun., 67:885-890, 1999.
Levitz, Trends Microbiol., 9:417-418, 2001.
Lin et al., Clin. Infect. Dis., 32:358-366, 2001.
Luberto et al., Genes Dev., 15:201-212, 2001.
Luberto et al., J. Clin. Invest. 112:1080-1094, 2003.
Luna, Histopathologic Methods and Color Atlas of Special Stains and Tissue Artifacts, 49-451, American Histolabs, Inc., MD, 1992.
Ma et al., Curr. Biol., 16:2156-60, 2006.
Mandala et al., J. Antibiot. (Tokyo), 48:349-356, 1995.
Mar et al., Clin. Infect. Dis., 34:909-917, 2002.
Marks et al., J. Biol. Chem., 276:26492-26498, 2001.
Marr, Curr. Opin. Hematol., 10:445-450, 2003.
Matsubara et al., Chem. Phys. Lipids, 43:1-12, 1987.
McNeil et al., Clin. Infect. Dis., 33:641-647, 2001.
Merrill et al., Methods, 36:207-224, 2005.
Mogensen et al., Cell, 5:103-111, 2006.
Mukherjee et al., Clin. Microbiol. Rev., 18:163-194, 2005.
National Institutes of Health, NHLBI/WHO Workshop Report, Bethesda, Md., 1995.
Negroni et al., Rev. Inst. Med. Trop. Sao Paulo, 37:385-389, 1995.
Nimrichter et al., Microbes Infect., 6:657-65, 2004.
Nimrichter et al., Microbes Infect., 6:657-665, 2004.
Perfect and Casadevall, In: Molecular principles of fungal pathogenesis, 1:3-11, Heitman et al. (ed.), ASM Press, Washington, D.C., 2006.
Pinto et al., Glycobiology, 12:251-260, 2002.
Rapp, Pharmacotherapy, 24:4 S-28S; quiz 29S-32S, 2004.
Raventos et al., Comb. Chem. High Throughput Screen, 8:219-233, 2005.
Rees et al., Clin. Infect. Dis., 27:1138-1147, 1998.
Rittershaus et al., J. Clin. Invest., 116:1651-59, 2006.
Rodrigues et al., Infect. Immun., 68:7049-7060, 2000.
Saito et al., Appl. Microbiol. Biotechnol., 69:1-7, 2006.
Sambrook et al., Molecular Cloning: A Laboratory Manual (Cold Spring Harbor Laboratory Press, Cold Spring Harbor, N.Y., 1989.
Santangelo et al., Infect. Immun., 72:2229-39, 2004.
Schwartz et al., Blood, 106:2641-2645, 2005.
Shaariah et al., Transplantation proceedings, 24:1898-1899, 1992.
Shaka et al., J. Magn. Reson., 77:274-293, 1988.
Shao et al., J. Immunol., 175:3244-51, 2005.
Shayman and Abe, Methods Enzymol., 311:42-49, 2000.
Shea et al., Infect. Immun., 74:xxx-yyy, 2006.
Sheehan and Hrapchak, Theory and Practice of Histotechnology, 196-197, The C.V. Mosby Company, London, 1980.
Speed et al., J. Med. Vet. Mycol., 34:187-193, 1996.
Steenbergen et al., Proc. Natl. Acad. Sci. USA, 98:15245-15250, 2001.
Stevens, J. Antimicrob. Chemother., 49 (Suppl 1):11-19, 2002.
Sullards et al., J. Mass. Spectrom., 35:347-353, 2000.
Takahashi et al., Braz. J. Med. Biol. Res., 29:1441-1444, 1996.
Thevissen et al., J. Biol. Chem., 279:3900-3905, 2004.
Toffaletti et al., J. Bacteriol., 175:1405-1411, 1993.
Toledo et al., Biochem. Biophys. Res. Commun., 280:19-24, 2001.
Toledo et al., Biochemistry, 38:7294-7306, 1999.
Toledo et al., Glycobiology, 11:105-112, 2001.
Toledo et al., Glycobiology, 11:113-124, 2001.
Toledo et al., J. Lipid Res., 41:797-806, 2000.
Toledo et al., J. Med. Vet. Mycol., 33:247-251, 1995.
Tucke et al., Proc. Natl. Acad. Sci. USA, 99:3165-70, 2002.
Tucker and Casadevall, Proc. Natl. Acad. Sci. USA, 99:3165-3170, 2002.
van Elden et al., Aids, 14:2787-2788, 2000.
Van Rooijen, Immunol. Methods, 1994; 174:83-93, 1994.
Villee, Biology, W. B. Saunders Co., Philadelphia and London, 1957.
Wang et al., Immunol. Rev., 157:53-60, 1997.
Wang et al., Proc. Natl. Acad. Sci. USA, 91:9402-6, 1994.
Warnecke and Heinz, Cell. Mol. Life. Sci., 60:919-941, 2003.
Warnecke et al., Cell. Mol. Life. Sci., 60:919-941, 2003.
Warny et al., Infect. Immun., 62:384-389, 1994.
White et al., J. Infect. Dis., 165:960-963, 1992.
Yeo and Wong, Clin. Microbiol. Rev., 15:465-484, 2002.

1. A method for detecting a pulmonary fungal infection prior to dissemination to the brain of an infected patient comprising:
(a) obtaining a blood sample from said patient; and
(b) assaying said sample for the presence of antibodies to fungal glucosylceramide,
wherein the presence of said antibodies indicates pulmonary fungal infection.

2. The method of claim 1, wherein said patient has been diagnosed with cancer or HIV infection, is being hospitalized in an intensive care unit, or is receiving immunosuppressive drugs.

3. The method of claim 1, wherein assaying comprises binding of antibodies to glucosylceramide fixed to a support.

4. The method of claim 3, wherein bound antibodies are detected using labeled anti-Ig antibodies.

5. The method of claim 4, wherein anti-Ig are labeled with an enzyme, a fluorescent label, a chemilluminescent label or a radiolabel.

6. The method of claim 1, wherein said blood sample is whole blood, serum or plasma.

7. The method of claim 1, further comprising treating said patient for fungal infection.

8. The method of claim 1, further comprising culturing a fungal sample from said subject.

9. The method of claim 8, wherein said fungal sample is typed to be Cryptococcus neoformans, Histoplasma capsulatum, Coccidioides immitis, Paracoccidioides brasiliensis, Bastomyces dermatitidis, Sporotrix schenckii, or Aspergillus fumigatus.

10. The method of claim 1, wherein said patient is asymptomatic at the time of assay.

11. A method of assessing the treatment of a patient for fungal infection comprising:
(a) obtaining a blood sample from said patient; and
(b) assaying said sample for the presence and/or level of antibodies to fungal glucosylceramide,
wherein the absence or presence of low levels of said antibodies indicates an effective treatment.

12. (canceled)

13. The method of claim 11, wherein assaying comprises binding of antibodies to glucosylceramide fixed to a support.

14. The method of claim 13, wherein antibodies are detected using labeled anti-Ig antibodies.

15. The method of claim 14, wherein anti-Ig are labeled with an enzyme, a fluorescent label, a chemilluminescent label or a radiolabel.

16. The method of claim 11, wherein said blood sample is whole blood, serum or plasma.

17. The method of claim 11, wherein said treatment is an antifungal mono- or combination therapy.

18. The method of claim 11, wherein steps (a) and (b) are repeated at different time points during said treatment.

19. The method of claim 11, wherein said treatment is altered based on the result of step (b).

20. The method of claim 19, wherein altered comprises changing the dose of a drug or changing from one drug to a different drug.

21. A method for treating a fungal infection in a subject comprising administering to said patient a therapeutically effective amount of an IgM or IgG monoclonal antibody to fungal glucosylceramide.

22. (canceled)

23. The method of claim 21, wherein said fungal infection is caused by Cryptococcus neoformans, Histoplasma capsulatum, Coccidioides immitis, Paracoccidioides brasiliensis, Bastomyces dermatitidis, Sporotrix schenckii, or Aspergillus fumigatus.

24-26. (canceled)

27. A method for preventing or treating a fungal infection in a patient comprising administering to said patient in need thereof a therapeutically effective amount of a fungal glucosylceramide synthase inhibitor.

28. (canceled)

29. The method of claim 27, wherein said fungal infection is caused by Cryptococcus neoformans, Histoplasma capsulatum, Coccidioides immitis, Paracoccidioides brasiliensis, Bastomyces dermatitidis, Sporotrix schenckii, or Aspergillus fumigatus.

30. The method of claim 27, wherein said fungal glucosylceramide synthase inhibitor is a defensin or derivative thereof.

31. A method of reducing dissemination of a fungal infection in a patient comprising administering to said patient an agent that depletes alveolar macrophages

32-37. (canceled)
